(19) United States
(12) Patent Application Publication
(10) Pub. No.: US 2009/0123414 A1
(43) Pub. Date: May 14, 2009

(54) Il-15 Antigen Arrays And Uses Thereof

(75) Inventors: Martin F. Bachmann, Seuzach (CH); Patrik Maurer, Winterthur (CH); Yu Zou, Birmensdorf (CH); Alain Tissot, Zurich (CH)

Correspondence Address: STERNE, KESSLER, GOLDSTEIN & FOX P.L.L.C., 1100 NEW YORK AVENUE, N.W., WASHINGTON, DC, 20005, US

(73) Assignee: CYTOS BIOTECHNOLOGY AG, SCHIIEREN (CH)

(21) Appl. No.: 11/792,749

(22) PCT Filed: Dec. 12, 2005
(86) PCT No.: PCT/EP2005/056680

§ 371 Date: Jun. 11, 2007

Related U.S. Application Data
(60) Provisional application No. 60/635,179, filed on Dec. 13, 2004.

(51) Int. Cl. A61K 48/00; A61K 39/20

(52) U.S. Cl. 424/85.2; 424/93.2; 977/803

(57) ABSTRACT

The present invention is related to the fields of molecular biology, virology, immunology and medicine. The invention provides a composition comprising an ordered and repetitive antigen array, wherein the antigen is an IL-15 protein, an IL-15 mutein or an IL-15 fragment. More specifically, the invention provides a composition comprising a virus-like particle, and at least one IL-15 protein, IL-15 mutein or at least one IL-15 fragment linked thereto. The invention also provides a process for producing the composition. The compositions of the invention are useful in the production of vaccines for the treatment of inflammatory and chronic autoimmune diseases. The composition of the invention efficiently induces immune responses, in particular antibody responses. Furthermore, the compositions of the invention are particularly useful to efficiently induce self-specific immune responses within the indicated context.

### BACKGROUND OF THE INVENTION

1. Field of the Invention

The present invention is in the fields of medicine, public health, immunology, molecular biology and virology. The invention provides composition comprising a virus-like particle (VLP) and at least one antigen, wherein said antigen is an IL-15 protein, an IL-15 mutein or an IL-15 fragment linked to the VLP respectively.

The invention also provides a process for producing the composition. The compositions of this invention are useful in the production of vaccines, in particular, for the treatment of diseases in which IL-15 mediates, or contributes to the condition, particularly for the treatment of inflammatory and/or chronic autoimmune diseases. Moreover, the compositions of the invention induce efficient immune responses, in particular antibody responses. Furthermore, the compositions of the invention are particularly useful to efficiently induce self-specific immune responses within the indicated context.

2. Related Art

3. Background

Interleukin-15 (IL-15) is a pro-inflammatory cytokine, a glycoprotein of 14-15 kD that is structurally and functionally related to IL-2 (Tagaya et al., Immunity, 1996; 4:329-336). IL-15 binds and signals through a heterotrimeric receptor consisting of γ chain (γc), IL-2RP, and IL-15Rα. IL-2, IL-4, IL-7, IL-9, IL-15 and IL-21 all utilize receptors containing the γ chain, while IL-2 and IL-15 receptors also share IL-2RP. IL-15 is found currently to be the only cytokine that binds to IL-15Rα. IL-15 binds to IL-15 Rα alone with high affinity (Ka=1×10¹¹M⁻¹) and binds to IL-2Rβ and γ chain complex with intermediate affinity (Ka=1×10⁹M⁻¹).

Constitutive expression of IL-15 has been reported in various cells and tissues including monocytes, macrophages, fibroblasts, keratinocytes and dendritic cells (Waldmann and Tagaya, Annu Rev Immunol. 1999; 17:19-49; Fehniger and Caligiuri, Blood. 2001; 97:14-32). The expression is upregulated under inflammatory conditions, as reported for monocytes stimulated with IFN-γ and LPS or by infection with viruses, bacteria or protozoans (Kirman et al., Inflamm Res. 1998; 47:285-9; Waldmann et al., Int Rev Immunol. 1998; 16:205-26. Waldmann and Tagaya, Annu Rev Immunol. 1999; 17:19-49, Fehniger and Caligiuri, Blood. 2001; 97:14-32). Furthermore, in chronic inflammatory diseases such as rheumatoid arthritis, locally produced IL-15 is likely to amplify inflammation by the recruitment and activation of synovial T-cells. This IL-15-induced effect has been suggested to play a role in disease pathogenesis (Kirman et al., Inflamm Res. 1998; 47:28-9.; McInnes et al., Nat. Med. 1996; 2:175-82.; McInnes et al., Nat. Med. 1997; 3:189-95; Mclnnes and Liew, Immunol Today. 1998; 19:75-9.; Fehniger and Caligiuri, Blood. 2001; 97:14-32.).

Monoclonal antibodies specifically against IL-15 have been proposed in treating a number of chronic inflammatory diseases and/or autoimmune diseases. WO0002582 has disclosed of using IL-15 monoclonal antibody to treat inflammatory bowel disease. WO03017935 has disclosed of using IL-15 monoclonal antibody to inhibit IL-15 induced proinflammatory effects, in particular to treat psoriasis and arthritis.

Since the half life of a monoclonal antibody is only about two to four weeks in human body, shortcomings of monoclonal antibody therapy thus include the need for repeated injections of large amounts of antibody (Kaplan, Curr Opin Invest. Drugs. 2002; 3:1017-23.). High doses of antibodies can lead to side-effects such as infusion disease. Anti-antibodies can also be generated in patients in an allotypic response, even if human or humanized antibodies are used, leading to a decreased therapeutic effect or potentially causing side-effects. Moreover, the expense associated with the high production cost of humanized monoclonal antibody and with the need for frequent hospital visit renders this antibody treatment unavailable to many patients in need.

### SUMMARY OF THE INVENTION

We have, now, surprisingly found that the inventive compositions and vaccines, respectively, comprising at least one IL-15 protein, at least one IL-15 mutein or at least one IL-15 fragment, are capable of inducing strong immune responses, in particular strong antibody responses, leading to high antibody titer against the self-antigen IL-15. Moreover, we have surprisingly found that inventive compositions and vaccines, respectively, are capable of inducing strong immune responses, in particular strong antibody responses, with protective and/or therapeutic effect against the induction and development of inflammatory and/or chronic autoimmune diseases in which IL-15 plays a crucial role, such as rheumatoid arthritis. Furthermore, we have surprisingly found that the inventive compositions and vaccines, respectively, are capable of inducing strong immune responses, in particular strong antibody responses, with protective and/or therapeutic effect against the induction and development of atherosclerosis. This indicates that the immune responses, in particular the antibodies generated by the inventive compositions and vaccines, respectively, are, thus, capable of specifically recognizing IL-15 in vivo, and interfere with its function.

Thus, in the first aspect, the present invention provides a composition which comprises (a) a virus-like particle (VLP) with at least one first attachment site; and (b) at least one antigen with at least one second attachment site, wherein said at least one antigen is an IL-15

protein, an IL-15 mutein or an IL-15 fragment and wherein (a) and (b) are linked through said at least one first and said at least one second attachment site, preferably to form an ordered and repetitive antigen array. In preferred embodiments of the invention, the virus-like particles suitable for use in the present invention comprises recombinant protein, preferably recombinant coat protein, mutants or fragments thereof, of a virus, preferably of a RNA bacteriophage.

In one preferred embodiment, the inventive composition comprises at least one IL-15 mutein. IL-15 mutein does not have the biological activity of IL-15 while preferably retaining almost identical protein structure as IL-15. IL-15 is a potent T cell stimulating cytokine. Thus, the inventive composition comprising IL-15 mutein provides therapeutically effective medicine while typically avoiding introducing biologically active IL-15 into the body.

In another preferred embodiment, the inventive composition comprises at least one IL-15 fragment, wherein the fragment comprises at least one antigenic site of IL-15. While ensuring a strong and protective immune response, in particular an antibody response, the use of IL-15 fragments for the present invention may reduce a possible induction of self-specific cytotoxic T cell responses.

In another aspect, the present invention provides a vaccine composition. Furthermore, the present invention provides a method to administering the vaccine composition to a human or an animal, preferably a mammal. The inventive vaccine composition is capable of inducing strong immune response, in particular antibody response, without the presence of at least one adjuvant. Thus, in one preferred embodiment, the vaccine is devoid of an adjuvant. The avoidance of using adjuvant may reduce a possible occurrence of unwanted inflammatory T cell responses.

In one preferred embodiment, the VLP of the invention comprised by the composition and the vaccine composition, respectively, is recombinantly produced in a host and the VLP of a RNA phage is essentially free of host RNA or host DNA, preferably host nucleic acid. It is advantageous to reduce, or preferably to eliminate, the amount of host RNA or host DNA, preferably nucleic acid, to avoid unwanted T cell responses as well as other unwanted side effects, such as fever.

In one aspect, the present invention provides a method of treating atherosclerosis, asthma, or inflammatory and/or autoimmune disease, in which IL-15 protein mediates, or contributes to the condition, wherein the method comprises administering the inventive composition or the invention vaccine composition, respectively, to an animal or a human. Inflammatory and/or autoimmune diseases, in which IL-15 protein mediates, or contributes to the condition, are, for example but not limited to, rheumatoid arthritis, psoriatic arthritis, juvenile idiopathic arthritis, psoriasis, Crohn diseases.

In a further aspect, the present invention provides a pharmaceutical composition comprising the inventive composition and an acceptable pharmaceutical carrier.

In again a further aspect, the present invention provides for a method of producing the composition of the invention comprising (a) providing a VLP with at least one first attachment site; (b) providing at least one antigen, wherein said antigen is an IL-15 protein, an IL-15 mutein or an IL-15 fragment, with at least one second attachment site; and (c) combining said VLP and said at least one antigen to produce said composition, wherein said at least one antigen and said VLP are linked through said at least one first and said at least one second attachment sites.

### BRIEF DESCRIPTION OF THE FIGURES

FIG. 1 shows average clinical scores of arthritis in mice immunized with Qβ VLP-IL-15. FIG. 1A shows average clinical scores of arthritis of mice immunized with 50 μg Qβ VLP-IL-15 and of mice received PBS only. FIG. 1B shows average clinical scores of arthritis of mice immunized with 25 μg Qβ VLP-IL-15 and of mice immunizes with Qβ only. The bar is drawn at the mean score in each vaccinated group.

FIG. 2 shows the quantification and statistical analysis of the atherosclerotic plaque load in Apoe^{-/-} mice. Bars show mean atherosclerotic plaque load in percentage in the aorta of Apoe−/− mice immunized with Qβ-IL-15 (black bar) or with Qβ (white bar). Error bars show the standard error of the mean.

### DETAILED DESCRIPTION OF THE INVENTION

Antigen: As used herein, the term “antigen” refers to a molecule capable of being bound by an antibody or a T cell receptor (TCR) if presented by MHC molecules. The term “antigen”, as used herein, also encompasses T-cell epitopes. An antigen is additionally capable of being recognized by the immune system and/or being capable of inducing a humoral immune response and/or cellular immune response leading to the activation of B- and/or T-lymphocytes. This may, however, require that, at least in certain cases, the antigen contains or is linked to a Th cell epitope and is given in adjuvant. An antigen can have one or more epitopes (B- and T-epitopes). The specific reaction referred to above is meant to indicate that the antigen will preferably react, typically in a highly selective manner, with its corresponding antibody or TCR and not with the multitude of other antibodies or TCRs which may be evoked by other antigens. Antigens as used herein may also be mixtures of several individual antigens.

Antigenic site: The term “antigenic site” and the term “antigenic epitope”, which are used herein interchangeably, refer to continuous or discontinuous portions of a polypeptide, which can be bound immunospecifically by an antibody or by a T-cell receptor within the context of an MHC molecule. Immunospecific binding excludes non-specific binding but does not necessarily exclude cross-reactivity. Antigenic site typically comprise 5-10 amino acids in a spatial conformation which is unique to the antigenic site.

Associated: The term “associated” (or its noun association) as used herein refers to all possible ways, preferably chemical interactions, by which two molecules are joined together. Chemical interactions include covalent and non-covalent interactions. Typical examples for non-covalent interactions are ionic interactions, hydrophobic interactions or hydrogen bonds, whereas covalent interactions are based, by way of example, on covalent bonds such as ester, ether, phosphoester, amide, peptide, carbon-phosphorus bonds, carbon-sulfur bonds such as thioether, or imide bonds.

Attachment Site, First: As used herein, the phrase “first attachment site” refers to an element which is naturally occurring with the VLP or which is artificially added to the VLP, and to which the second attachment site may be linked. The first attachment site may be a protein, a polypeptide, an amino acid, a peptide, a sugar, a polynucleotide, a natural or synthetic polymer, a secondary metabolite or compound (biotin, fluorescein, retinol, digoxigenin, metal ions, phenylmethylsulfonylfluoride), or a chemically reactive group such as an amino group, a carboxyl group, a sulfhydryl group, a hydroxyl group, a guanidinyl group, histidinyl group, or a combination thereof A preferred embodiment of a chemically reactive group being the first attachment site is the amino group of an amino acid such as lysine. The first attachment site is located, typically on the surface, and preferably on the outer surface of the VLP. Multiple first attachment sites are present on the surface, preferably on the outer surface of virus-like particle, typically in a repetitive configuration. In a preferred embodiment the first attachment site is associated with the VLP, through at least one covalent bond, preferably through at least one peptide bond.

Attachment Site, Second: As used herein, the phrase “second attachment site” refers to an element which is naturally occurring with or which is artificially added to the IL-15 of the invention and to which the first attachment site may be linked. The second attachment site of IL-15 of the invention may be a protein, a polypeptide, a peptide, an amino acid, a sugar, a polynucleotide, a natural or synthetic polymer, a secondary metabolite or compound (biotin, fluorescein, retinol, digoxigenin, metal ions, phenylmethylsulfonylfluoride), or a chemically reactive group such as an amino group, a carboxyl group, a sulfhydryl group, a hydroxyl group, a guanidinyl group, histidinyl group, or a combination thereof. A preferred embodiment of a chemically reactive group being the second attachment site is the sulfhydryl group, preferably of an amino acid cysteine. The terms “IL-15 protein with at least one second attachment site”, “IL-15 mutein with at least one second attachment site”, “IL-15 fragment with at least one second attachment site” or “IL-15 of the invention with at least one second attachment site” refer, therefore, to a construct comprising the IL-15 of the invention and at least one second attachment site. However, in particular for a second attachment site, which is not naturally occurring with the IL-15 protein, IL-15 mutein or the IL-15 fragment, such a construct typically and preferably further comprises a “linker”. In another preferred embodiment the second attachment site is associated with the IL-15 of the invention through at least one covalent bond, preferably through at least one peptide bond. In yet another preferred embodiment, the second attachment site is artificially added to the IL-15 of the invention through a linker, preferably comprising a cysteine. Preferably the linker is fused to the IL-15 of the invention by a peptide.

Coat protein: The term “coat protein” and the interchangeably used term “capsid protein” within this application, refers to a viral protein, preferably a subunit of a natural capsid of a virus, preferably of a RNA-phage, which is capable of being incorporated into a virus capsid or a VLP. Typically and preferably the term “coat protein” refers to the coat protein encoded by the genome of a virus, preferably an RNA bacteriophage or by the genome of a variant of a virus, preferably of an RNA bacteriophage. More preferably and by way of example, the term “coat protein of AP205” refers to SEQ ID NO: 14 or the amino acid sequence, wherein the first methionine is cleaved from SEQ ID NO: 14. More preferably and by way of example, the term “coat protein of Qβ” refers to SEQ ID NO: 1 (“Qβ CP”) and SEQ ID NO:2 (A1), with or without the methione at the N-terminus. The capsid of bacteriophage Qβ is composed mainly of the Qβ CP, with a minor content of the A1 protein.

IL-15 of the invention: The term “IL-15 of the invention” as used herein, refers to at least one IL-15 protein, at least one IL-15 mutein or at least one IL-15 fragment as defined herein or any combination thereof.

IL-15 protein: The term “IL-15 protein” as used herein should encompass any polypeptide comprising, or alternatively or preferably consisting of, the human IL-15 of SEQ ID NO:23, the mouse IL-15 of SEQ ID NO:24, the rat IL-15 of SEQ ID NO:25 or the corresponding orthologs from any other animal. Moreover, the term “IL-15 protein” as used herein should also encompass any polypeptide comprising, or alternatively or preferably consisting of, any natural or genetically engineered variant having more than 70%, preferably more than 80%, preferably more than 85%, even more preferably more than 90%, again more preferably more than 95%, and most preferably more than 97% amino acid sequence identity with the human IL-15 of SEQ ID NO:23, the mouse IL-15 of SEQ ID NO:24, the rat IL-15 of SEQ ID NO:25 or the corresponding orthologs from any other animal. The term “IL-15 protein” as used herein should furthermore encompass post-translational modifications including but not limited to glycosylations, acetylations, phosphorylations of the IL-15 protein as defined above. Preferably the IL-15 protein, as defined herein, consists of at most 500 amino acids in length, and even more preferably of at most 300 amino acids in length, still preferably at most 200 amino acids in length and still further preferably at most 150, still further preferably at most 130 amino acids in length. Typically and preferably, IL-15 protein is capable of inducing in vivo the production of antibody specifically binding to IL-15, as verified by, for example ELISA.

IL-15 mutein: The term “IL-15 mutein” as used herein, should encompass any polypeptide that is IL-15 protein and said polypeptide does not have IL-15 biological activity. More preferably, the term “IL-15 mutein” refers to any polypeptide that differs from the human IL-15 of SEQ ID NO:23, the mouse IL-15 of SEQ ID NO:24, the rat IL-15 of SEQ ID NO:25 or the corresponding orthologs from any other animal by at least one and by at most six, preferably at most five, more preferably at most four, more preferably at most three, even more preferably at most two, most preferably one amino acid and said polypeptide does not have IL-15 biological activity. Typically and preferably, the composition of the invention comprising an IL-15 mutein is capable of inducing in vivo the production of antibody specifically binding to IL-15. The term “IL-15 biological activity” as used herein, refers to the capability of stimulating T-lymphocytes proliferation and/or differentiation.

A typical and the preferred assay for measuring IL-15 biological activity has been disclosed in EXAMPLE 2 in EP 0772624 and is incorporated herein by way of reference. An IL-15 protein is tested in the same experiment with the corresponding wild type IL-15 used as a positive control. The corresponding wild type IL-15 refers to the IL-15 that is of the same species as the IL-15 protein. Protein concentration assay, for example, Bradford assay, is performed to ensure that stochiometrically equal amounts of mutant of IL-15 protein and its corresponding wild type IL-15 used as a positive control are tested in the same experiment. It is considered as equal amount if the amount of IL-15 to-be-tested and the amount of the corresponding wild type IL-15 used as a positive control are not different from each other by more than 3%, preferably by more than 1%.

A particular IL-15 protein does not have IL-15 biological activity if it has at most 20%, preferably 10%, more preferably 5%, even more preferably 1%, still more preferably 0.2% of the IL-15 biological activity of equal amount of the corresponding wild type IL-15 used as a positive control.

IL-15 fragment: The term “IL-15 fragment” as used herein should encompass any polypeptide comprising, or alternatively or preferably consisting of, at least 4, 5, 6, 7, 8, 9, 10, 11, 12, 17, 18, 19, 20, 25, 30 contiguous amino acids of a IL-15 protein or IL-15 mutein as defined herein as well as any polypeptide having more than 65%, preferably more than 80%, more preferably 85%, more preferably more than 90% and even more preferably more than 95% amino acid sequence identity thereto. Preferably, the term “IL-15 fragment” as used herein should encompass any polypeptide comprising, or alternatively or preferably consisting of, at least 6 contiguous amino acids of an IL-15 protein or an IL-15 mutein as defined herein as well as any polypeptide having more than 80%, more than 85%, preferably more than 90% and even more preferably more than 95% amino acid sequence identity thereto. Preferred embodiments of IL-15 fragment are truncation or internal deletion forms of IL-15 protein. or IL-15 mutein. Typically and preferably, an IL-15 fragment is capable of inducing the production of antibody in vivo, which specifically binds to IL-15.

The amino acid sequence identity of polypeptides can be determined conventionally using known computer programs such as the Bestfit program. When using Bestfit or any other sequence alignment program, preferably using Bestfit, to determine whether a particular sequence is, for instance, 95% identical to a reference amino acid sequence, the parameters are set such that the percentage of identity is calculated over the full length of the reference amino acid sequence and that gaps in homology of up to 5% of the total number of amino acid residues in the reference sequence are allowed. This aforementioned method in determining the percentage of identity between polypeptides is applicable to all proteins, polypeptides or a fragment thereof disclosed in this invention.

Linked: The term “linked” (or its noun: linkage) as used herein, refers to all possible ways, preferably chemical interactions, by which the at least one first attachment site and the at least one second attachment site are joined together. Chemical interactions include covalent and non-covalent interactions. Typical examples for non-covalent interactions are ionic interactions, hydrophobic interactions or hydrogen bonds, whereas covalent interactions are based, by way of example, on covalent bonds such as ester, ether, phosphoester, amide, peptide, carbon-phosphorus bonds, carbon-sulfur bonds such as thioether, or imide bonds. In certain preferred embodiments the first attachment site and the second attachment site are linked through at least one covalent bond, preferably through at least one non-peptide bond, and even more preferably through exclusively non-peptide bond(s). The term “linked” as used herein, however, shall not only encompass a direct linkage of the at least one first attachment site and the at least one second attachment site but also, alternatively and preferably, an indirect linkage of the at least one first attachment site and the at least one second attachment site through intermediate molecule(s), and hereby typically and preferably by using at least one, preferably one, heterobifunctional cross-linker.

Linker: A “linker”, as used herein, either associates the second attachment site with IL-15 of the invention or already comprises, essentially consists of, or consists of the second attachment site. Preferably, a “linker”, as used herein, already comprises the second attachment site, typically and preferably—but not necessarily—as one amino acid residue, preferably as a cysteine residue. A “linker” as used herein is also termed “amino acid linker”, in particular when a linker according to the invention contains at least one amino acid residue. Thus, the terms “linker” and “amino acid linker” are interchangeably used herein. However, this does not imply that such a linker consists exclusively of amino acid residues, even if a linker consisting of amino acid residues is a preferred embodiment of the present invention. The amino acid residues of the linker are, preferably, composed of naturally occurring amino acids or unnatural amino acids known in the art, all-L or all-D or mixtures thereof Further preferred embodiments of a linker in accordance with this invention are molecules comprising a sulfhydryl group or a cysteine residue and such molecules are, therefore, also encompassed within this invention. Further linkers useful for the present invention are molecules comprising a C1-C6 alkyl-, a cycloalkyl such as a cyclopentyl or cyclohexyl, a cycloalkenyl, aryl or heteroaryl moiety. Moreover, linkers comprising preferably a C1-C6 alkyl-, cycloalkyl-(C5, C6), aryl- or heteroaryl- moiety and additional amino acid(s) can also be used as linkers for the present invention and shall be encompassed within the scope of the invention. Association of the linker with the IL-15 of the invention is preferably by way of at least one covalent bond, more preferably by way of at least one peptide bond.

Ordered and repetitive antigen array: As used herein, the term “ordered and repetitive antigen array” generally refers to a repeating pattern of antigen or, characterized by a typically and preferably high order of uniformity in spacial arrangement of the antigens with respect to virus-like particle, respectively. In one embodiment of the invention, the repeating pattern may be a geometric pattern. Certain embodiments of the invention, such as VLP of RNA phages, are typical and preferred examples of suitable ordered and repetitive antigen arrays which, moreoever, possess strictly repetitive paracrystalline orders of antigens, preferably with spacings of 1 to 30 nanometers, preferably 2 to 15 nanometers, even more preferably 2 to 10 nanometers, even again more preferably 2 to 8 nanometers, and further more preferably 1.6 to 7 nanometers.

Packaged: The term “packaged” as used herein refers to the state of a polyanionic macromolecule in relation to the VLP. The term “packaged” as used herein includes binding that may be covalent, e.g., by chemically coupling, or non-covalent, e.g., ionic interactions, hydrophobic interactions, hydrogen bonds, etc. The term also includes the enclosement, or partial enclosement, of a polyanionic macromolecule. Thus, the polyanionic macromolecule can be enclosed by the VLP without the existence of an actual binding, in particular of a covalent binding. In preferred embodiments, the at least one polyanionic macromolecule is packaged inside the VLP, most preferably in a non-covalent manner.

Polypeptide: The term “polypeptide” as used herein refers to a molecule composed of monomers (amino acids) linearly linked by amide bonds (also known as peptide bonds). It indicates a molecular chain of amino acids and does not refer to a specific length of the product. Thus, peptides, dipeptides, tripeptides, oligopeptides and proteins are included within the definition of polypeptide. Post-translational modifications of the polypeptide, for example, glycosylations, acetylations, phosphorylations, and the like are also encompassed.

Virus particle: The term “virus particle” as used herein refers to the morphological form of a virus. In some virus types it comprises a genome surrounded by a protein capsid; others have additional structures (e.g., envelopes, tails, etc.).

Virus-like particle (VLP), as used herein, refers to a non-replicative or non-infectious, preferably a non-replicative and non-infectious virus particle, or refers to a non-replicative or non-infectious, preferably a non-replicative and non-infectious structure resembling a virus particle, preferably a capsid of a virus. The term “non-replicative”, as used herein, refers to being incapable of replicating the genome comprised by the VLP. The term “non-infectious”, as used herein, refers to being incapable of entering the host cell. Preferably a virus-like particle in accordance with the invention is non-replicative and/or non-infectious since it lacks all or part of the viral genome or genome function. In one embodiment, a virus-like particle is a virus particle, in which the viral genome has been physically or chemically inactivated. Typically and more preferably a virus-like particle lacks all or part of the replicative and infectious components of the viral genome. A virus-like particle in accordance with the invention may contain nucleic acid distinct from their genome. A typical and preferred embodiment of a virus-like particle in accordance with the present invention is a viral capsid such as the viral capsid of the corresponding virus, bacteriophage, preferably RNA-phage. The terms “viral capsid” or “capsid”, refer to a macromolecular assembly composed of viral protein subunits. Typically, there are 60, 120, 180, 240, 300, 360 and more than 360 viral protein subunits. Typically and preferably, the interactions of these subunits lead to the formation of viral capsid or viral-capsid like structure with an inherent repetitive organization, wherein said structure is, typically, spherical or tubular.

Virus-like particle of a RNA phage: As used herein, the term “virus-like particle of a RNA phage” refers to a virus-like particle comprising, or preferably consisting essentially of or consisting of coat proteins, mutants or fragments thereof, of a RNA phage. In addition, virus-like particle of a RNA phage resembling the structure of a RNA phage, being non replicative and/or non-infectious, and lacking at least the gene or genes encoding for the replication machinery of the RNA phage, and typically also lacking the gene or genes encoding the protein or proteins responsible for viral attachment to or entry into the host. This definition should, however, also encompass virus-like particles of RNA phages, in which the aforementioned gene or genes are still present but inactive, and, therefore, also leading to non-replicative and/or non-infectious virus-like particles of a RNA phage. Within this present disclosure the term “subunit” and “monomer” are interexchangeably and equivalently used within this context. In this application, the term “RNA-phage” and the term “RNA-bacteriophage” are interchangeably used.

One, a, or an: when the terms “one”, “a”, or “an” are used in this disclosure, they mean “at least one” or “one or more” unless otherwise indicated.

Within this application, antibodies are defined to be specifically binding if they bind to the antigen with a binding affinity (Ka) of 10⁶ M⁻¹ or greater, preferably 10⁷ M⁻¹ or greater, more preferably 10⁸ M⁻¹ or greater, and most preferably 10⁹ M⁻¹ or greater. The affinity of an antibody can be readily determined by one of ordinary skill in the art (for example, by Scatchard analysis.)

This invention provides compositions and methods for enhancing immune responses against IL-15 in an animal or in human. Compositions of the invention comprises: (a) a virus-like particle (VLP) with at least one first attachment site; and (b) at least one antigen with at least one second attachment site, wherein the at least one antigen is an IL-15 protein, an IL-15 mutein or an IL-15 fragment and wherein (a) and (b) are linked through the at least one first and the at least one second attachment site. Preferably, the IL-15 protein, the IL-15 mutein or the IL-15 fragment is linked to the VLP, so as to form an ordered and repetitive antigen-VLP array. In preferred embodiments of the invention, at least 20, preferably at least 30, more preferably at least 60, again more preferably at least 120 and further more preferably at least 180 IL-15 of the invention are linked to the VLP.

Any virus known in the art having an ordered and repetitive structure may be selected as a VLP of the invention. Illustrative DNA or RNA viruses, the coat or capsid protein of which can be used for the preparation of VLPs have been disclosed in WO 2004/009124 on page 25, line 10-21, on page 26, line 11-28, and on page 28, line 4 to page 31, line 4. These disclosures are incorporated herein by way of reference.

Virus or virus-like particle can be produced and purified from virus-infected cell culture. The resulting virus or virus-like particle for vaccine purpose needs to be devoid of virulence. A virulent virus or virus-like particle may be generated by chemical and/or physical inactivation, such as UV irradiation, formaldehyde treatment. Alternatively, the genome of the virus may be genetically manipulated by mutations or deletions to render the virus replication incompetent.

In one preferred embodiment, the VLP is a recombinant VLP. Almost all commonly known viruses have been sequenced and are readily available to the public. The gene encoding the coat protein can be easily identified by a skilled artisan. The preparation of VLPs by recombinantly expressing the coat protein in a host is within the common knowledge of a skilled artisan.

In one preferred embodiment, the virus-like particle comprises, or alternatively consists of, recombinant proteins, mutants or fragments thereof, of a virus selected form the group consisting of: a) RNA phages; b) bacteriophages; c) Hepatitis B virus, preferably its capsid protein (Ulrich, et al., Virus Res. 50:141-182 (1998)) or its surface protein (WO 92/11291); d) measles virus (Warnes, et al., Gene 160:173-178 (1995)); e) Sindbis virus; f) rotavirus (U.S. Pat. No. 5,071,651 and U.S. Pat. No. 5,374,426); g) foot-and-mouth-disease virus (Twomey, et al., Vaccine 13:1603 1610, (1995)); h) Norwalk virus (Jiang, X., et al., Science 250:1580 1583 (1990); Matsui, S. M., et al., J. Clin. Invest. 87:1456 1461 (1991)); i) Alphavirus; j) retrovirus, preferably its GAG protein (WO 96/30523); k) retrotransposon Ty, preferably the protein p1; 1) human Papilloma virus (WO 98/15631); m) Polyoma virus; n) Tobacco mosaic virus; and o) Flock House Virus.

In one preferred embodiment, the VLP comprises, or consists of, more than one amino acid sequence, preferably two amino acid sequences, of the recombinant proteins, mutants or fragments thereof VLP comprises or consists of more than one amino acid sequence is referred, in this application, as mosaic VLP.

The term “fragment of a recombinant protein” or the term “fragment of a coat protein”, as used herein, is defined as a polypeptide, which is of at least 70%, preferably at least 80%, more preferably at least 90%, even more preferably at least 95% the length of the wild-type recombinant protein, or coat protein, respectively and which preferably retains the capability of forming VLP. Preferably the fragment is obtained by at least one internal deletion, at least one truncation or at least one combination thereof. The term “fragment of a recombinant protein” or “fragment of a coat protein” shall further encompass polypeptide, which has at least 80%, preferably 90%, even more preferably 95% amino acid sequence identity with the “fragment of a recombinant protein” or “fragment of a coat protein”, respectively, as defined above and which is preferably capable of assembling into a virus-like particle.

The term “mutant recombinant protein” or the term “mutant of a recombinant protein” as interchangeably used in this invention, or the term “mutant coat protein” or the term “mutant of a coat protein”, as interchangeably used in this invention, refers to a polypeptide having an amino acid sequence derived from the wild type recombinant protein, or coat protein, respectively, wherein the amino acid sequence is at least 80%, preferably at least 85%, 90%, 95%, 97%, or 99% identical to the wild type sequence and preferably retains the ability to assemble into a VLP.

Assembly of the fragment or mutant of recombinant protein or coat protein into a VLP may be tested, as one skilled in the art would appreciate by expressing the protein in E.coli, optionally purifying the capsids by gel filtration from cell lysate, and analysing the capsid formation in an immunodiffusion assay (Ouchterlony test) or by Electron Microscopy (EM) (Kozlovska, T. M. et al., Gene 137:133-37 (1993)). Immunodiffusion assays and EM may be directly performed on cell lysate.

In one preferred embodiment, the virus-like particle of the invention is of Hepatitis B virus. The preparation of Hepatitis B virus-like particles have been disclosed, inter alia, in WO 00/32227, WO 01/85208 and in WO 01/056905. All three documents are explicitly incorporated herein by way of reference. Other variants of HBcAg suitable for use in the practice of the present invention have been disclosed in page 34-39 WO 01/056905.

In one further preferred embodiments of the invention, a lysine residue is introduced into the HBcAg polypeptide, to mediate the linking of IL-15 of the invention to the VLP of HBcAg. In preferred embodiments, VLPs and compositions of the invention are prepared using a HBcAg comprising, or alternatively consisting of, amino acids 1-144, or 1-149, 1-185 of SEQ ID NO:20, which is modified so that the amino acids at positions 79 and 80 are replaced with a peptide having the amino acid sequence of Gly-Gly-Lys-Gly-Gly. This modification changes the SEQ ID NO:20 to SEQ ID NO:21. In further preferred embodiments, the cysteine residues at positions 48 and 110 of SEQ ID NO:21, or its corresponding fragments, preferably 1-144 or 1-149, are mutated to serine. The invention further includes compositions comprising Hepatitis B core protein mutants having above noted corresponding amino acid alterations. The invention further includes compositions and vaccines, respectively, comprising HBcAg polypeptides which comprise, or alternatively consist of, amino acid sequences which are at least 80%, 85%, 90%, 95%, 97% or 99% identical to SEQ ID NO:21.

In another embodiment of the invention, the virus-like particle is a recombinant alphavirus, and more specifically, a recombinant Sindbis virus. Alphaviruses are positive stranded RNA viruses that replicate their genomic RNA entirely in the cytoplasm of the infected cell without a DNA intermediate (Strauss, J. and Strauss, E., Microbiol. Rev. 58:491-562 (1994)). Several members of the alphavirus family, Sindbis (Schlesinger, S., Trends Biotechnol. 11:18-22 (1993)), Semliki Forest Virus (SFV) (Liljestrom, P. & Garoff, H., Bio/Technology 9:1356-1361 (1991)) and others (Davis, N. L. et al., Virology 171:189-204 (1989)), have received considerable attention for use as virus-based expression vectors for a variety of different proteins (Lundstrom, K., Curr. Opin. Biotechnol. 8:578-582 (1997)) and as candidates for vaccine development.

In one preferred embodiment of the invention, the virus-like particle of the invention comprises, consists essentially of, or alternatively consists of, recombinant coat proteins, mutants or fragments thereof, of a RNA-phage. Preferably, the RNA-phage is selected from the group consisting of a) bacteriophage Qβ; b) bacteriophage R17; c) bacteriophage fr; d) bacteriophage GA; e) bacteriophage SP; f) bacteriophage MS2; g) bacteriophage M11; h) bacteriophage MX1; i) bacteriophage NL95; k) bacteriophage f2; l) bacteriophage PP7 and m) bacteriophage AP205.

In one preferred embodiment of the invention, the composition comprises coat protein, mutants or fragments thereof, of RNA phages, wherein the coat protein has amino acid sequence selected from the group consisting of: (a) SEQ ID NO:1: referring to Qβ CP; (b) a mixture of SEQ ID NO:1 and SEQ ID NO:2.(referring to Qβ A1 protein); (c) SEQ ID NO:3; (d) SEQ ID NO:4; (e) SEQ ID NO:5; (f) SEQ ID NO:6, (g) a mixture of SEQ ID NO:6 and SEQ ID NO:7; (h) SEQ ID NO:8; (i) SEQ ID NO:9; (j) SEQ ID NO:10; (k) SEQ ID NO:11; (l) SEQ ID NO:12; (m) SEQ ID NO:13; and (n) SEQ ID NO:14. Generally the coat protein mentioned above is capable of assembly into VLP with or without the presence of the N-terminal methionine.

In one preferred embodiment of the invention, the VLP is a mosaic VLP comprising or alternatively consisting of more than one amino acid sequence, preferably two amino acid sequences, of coat proteins, mutants or fragments thereof, of a RNA phage.

In one very preferred embodiment, the VLP comprises or alternatively consists of two different coat proteins of a RNA phage, said two coat proteins have an amino acid sequence of SEQ ID NO:1 and SEQ ID NO:2, or of SEQ ID NO:6 and SEQ ID NO:7.

In preferred embodiments of the present invention, the virus-like particle of the invention comprises, or alternatively consists essentially of, or alternatively consists of recombinant coat proteins, mutants or fragments thereof, of the RNA-bacteriophage Qβ, fr, AP205 or GA.

In one preferred embodiment, the VLP of the invention is a VLP of RNA-phage Qβ. The capsid or virus-like particle of Qβ showed an icosahedral phage-like capsid structure with a diameter of 25 nm and T=3 quasi symmetry. The capsid contains 180 copies of the coat protein, which are linked in covalent pentamers and hexamers by disulfide bridges (Golmohammadi, R. et al., Structure 4:543-5554 (1996)), leading to a remarkable stability of the Qβ capsid. Capsids or VLPs made from recombinant Qβ coat protein may contain, however, subunits not linked via disulfide bonds to other subunits within the capsid, or incompletely linked. The capsid or VLP of Qβ shows unusual resistance to organic solvents and denaturing agents. Surprisingly, we have observed that DMSO and acetonitrile concentrations as high as 30%, and guanidinium concentrations as high as 1 M do not affect the stability of the capsid. The high stability of the capsid or VLP of Qβ is an advantageous feature, in particular, for its use in immunization and vaccination of mammals and humans in accordance of the present invention.

Further preferred virus-like particles of RNA-phages, in particular of Qβ and fr in accordance of this invention are disclosed in WO 02/056905, the disclosure of which is herewith incorporated by reference in its entirety. Particular example 18 of WO 02/056905 gave detailed description of preparation of VLP particles from Qβ.

In another preferred embodiment, the VLP of the invention is a VLP of RNA phage AP205. Assembly-competent mutant forms of AP205 VLPs, including AP205 coat protein with the substitution of proline at amino acid 5 to threonine, may also be used in the practice of the invention and leads to other preferred embodiments of the invention. WO 2004/007538 describes, in particular in Example 1 and Example 2, how to obtain VLP comprising AP205 coat proteins, and hereby in particular the expression and the purification thereto. WO 2004/007538 is incorporated herein by way of reference. AP205 VLPs are highly immunogenic, and can be linked with IL-15 of the invention to typically and preferably generate vaccine constructs displaying the IL-15 of the invention oriented in a repetitive manner. High antibody titer is elicited against the so displayed IL-15 of the inventions showing that linked IL-15 of the inventions are accessible for interacting with antibody molecules and are immunogenic.

In one preferred embodiment, the VLP of the invention comprises or consists of a mutant coat protein of a virus, preferably a RNA phage, wherein the mutant coat protein has been modified by removal of at least one lysine residue by way of substitution and/or by way of deletion. In another preferred embodiment, the VLP of the invention comprises or consists of a mutant coat protein of a virus, preferably a RNA phage, wherein the mutant coat protein has been modified by addition of at least one lysine residue by way of substitution and/or by way of insertion. In one very preferred embodiment, the mutant coat protein is of RNA phage Qβ, wherein at least one, or alternatively at least two, lysine residue have been removed by way of substitution or by way of deletion. In an alternative very preferred embodiment, the mutant coat protein is of RNA phage Qβ, wherein at least one, or alternatively at least two, lysine residue have been added by way of substitution or by way of insertion. In one further preferred embodiment, the mutant coat protein of RNA phage Qβ has an amino acid sequence selected from any one of SEQ ID NO:15-19. The deletion, substitution or addition of at least one lysine residue allows varying the degree of coupling, i.e. the amount of IL-15 of the invention per subunits of the VLP of a virus, preferably of a RNA-phages, in particular, to match and tailor the requirements of the vaccine.

In one preferred embodiment, the compositions and vaccines of the invention have an antigen density being from 0.5 to 4.0. The term “antigen density”, as used herein, refers to the average number of IL-15 of the invention which is linked per subunit, preferably per coat protein, of the VLP, and hereby preferably of the VLP of a RNA phage. Thus, this value is calculated as an average over all the subunits or monomers of the VLP, preferably of the VLP of the RNA-phage, in the composition or vaccines of the invention.

In another preferred embodiment of the present invention, the virus-like particle comprises, or alternatively consists essentially of, or alternatively consists of mutant coat protein of Qβ, or mutants or fragments thereof, and the corresponding A1 protein. In a further preferred embodiment, the virus-like particle comprises, or alternatively consists essentially of, or alternatively consists of mutant coat protein with amino acid sequence SEQ ID NO:15, 16, 17, 18, or 19 and the corresponding A1 protein.

Assembly-competent mutant forms of AP205 VLPs, including AP205 coat protein with the substitution of proline at amino acid 5 to threonine, asparigine at amino acid 14 to aspartic acid, may also be used in the practice of the invention and leads to other preferred embodiments of the invention. The cloning of the AP205 Pro-5-Thr and the purification of the VLPs are disclosed in WO 2004/007538, and therein, in particular within Example 1 and Example 2. The disclosure of WO 2004/007538, and, in particular, Example 1 and Example 2 thereof is explicitly incorporated herein by way of reference.

Further RNA phage coat proteins have also been shown to self-assemble upon expression in a bacterial host (Kastelein, R A. et al., Gene 23:245-254 (1983), Kozlovskaya, T M. et al., Dokl. Akad. Nauk SSSR 287:452-455 (1986), Adhin, M R. et al., Virology 170:238-242 (1989), Priano, C. et al., J. Mol. Biol. 249:283-297 (1995)). In particular the biological and biochemical properties of GA (Ni, CZ., et al., Protein Sci. 5:2485-2493 (1996), Tars, K et al., J. Mol.Biol. 271:759-773(1997)) and of fr (Pushko P. et al., Prot. Eng. 6:883-891 (1993), Liljas, L et al. J Mol. Biol. 244:279-290, (1994)) have been disclosed. The crystal structure of several RNA bacteriophages has been determined (Golmohammadi, R. et al., Structure 4:543-554 (1996)). Using such information, surface exposed residues can be identified and, thus, RNA-phage coat proteins can be modified such that one or more reactive amino acid residues can be inserted by way of insertion or substitution. Another advantage of the VLPs derived from RNA phages is their high expression yield in bacteria that allows production of large quantities of material at affordable cost.

In one preferred embodiment, the composition of the invention comprises at least one antigen, wherein said at least one antigen is an IL-15 protein, an IL-15 fragment, or an IL-15 mutein. In one preferred embodiment, the IL-15 protein, the IL-15 mutein or the IL-15 fragment is selected from a origin selected from the group consisting of: (a) human origin; (b) bovine origin; (c) sheep origin; (d) dog origin; (e) feline origin; (f) mouse origin; (g) pig origin; (h) chicken origin (i) horse origin; and (j) rat origin.

In one preferred embodiment, the at least one antigen is an IL-15 protein. In a further preferred embodiment, the IL-15 protein comprises or consists of an amino acid sequence selected from the consisting of: (a) SEQ ID NO:22; (b) SEQ ID NO:23; (c) SEQ ID NO:24; (d) SEQ ID NO:25; and (e) an amino acid sequence which is at least 80%, or preferably at least 85%, more preferably at least 90%, or most preferably at least 95% identical with any of SEQ ID NOs: 22-25.

In another preferred embodiment, the at least one antigen is an IL-15 mutein. IL-15 mutein does not have IL-15 biological activity, yet is capable of inducing antibody responses specifically against IL-15. Therefore using IL-15 mutein as the antigen in accordance with the present invention ensures the avoidance of, however, unexpected and undesired side effect due to the introduction of IL-15 coupled to VLP in accordance with the present invention. In U.S. Pat. No. 6,013,480 two muteins have been disclosed which are capable of binding to the IL-15 R α-subunit and incapable of transducing a signal through the β- or γ-subunits of the IL-15 receptor complex. Muteins which are not biological active and incapable of binding to the α-subunit have also been disclosed (Bernard J. et al. J Biol Chem. (2004);279(23): 24313-22). Therefore, in one preferred embodiment, IL-15 mutein comprises or consists of an amino acid sequence selected from a group consisting of: (a) SEQ ID NO:23, wherein position 46 is not E; (b) SEQ ID NO:23, wherein position 50 is not I; (c) SEQ ID NO:23, wherein position 46 is not E and position 50 is not I; (d) SEQ ID NO:31; (e) SEQ ID NO:32; (f) SEQ ID NO:33; (g) an amino acid sequence which is at least 80%, or preferably at least 85%, more preferably at least 90%, or most preferably at least 95% identical with SEQ ID NO:23, wherein the position corresponding to position 46 of SEQ ID NO:23 is not E, or the position corresponding to position 50 of SEQ ID NO:23 is not I, or the position corresponding to position 46 of SEQ ID NO:23 is not E and the position corresponding to position 50 of SEQ ID NO:23 is not I; (h) SEQ ID NO:23, wherein either or both amino acid residues Asp⁸ or Gln¹⁰⁸ either is deleted or is substituted with a different naturally-occurring amino acid; (i) SEQ ID NO:23, wherein either or both amino acid residues Gln¹⁰¹ or Gln¹⁰⁸ either is deleted or is substituted with a different naturally-occurring amino acid; (j) SEQ ID NO:42; (j) SEQ ID NO:23, wherein position 8 is not Asp, preferably not Asp or Glu; (k) SEQ ID NO:23, wherein either or both of Asp⁸ or Gln¹⁰⁸ is each substituted with a serine or cysteine; (l) SEQ ID NO:23, wherein at least one amino acid at position out of 8, 101 and 108 is deleted or preferably substituted.

In one further preferred embodiment, the IL-15 mutein comprises or consists of amino acid sequence of SEQ ID NO:23, wherein position 46 is not Glu; Asp, Gln or Asn. In a still further preferred embodiment, the IL-15 mutein comprises or consists of an amino acid sequence of SEQ ID NO:31.

In one further preferred embodiment, the IL-15 mutein comprises or consists of amino acid sequence of SEQ ID NO:23, wherein position 50 is not Ile or Leu. In a further preferred embodiment, the IL-15 has amino acid sequence, wherein position 50 is not Ile, Leu, Ala, Gly or Val. In a still further preferred embodiment, the IL-15 mutein comprises or consists of an amino acid sequence of SEQ ID NO:32.

In one further preferred embodiment, the IL-15 mutein comprises or consists of an amino acid sequence of SEQ ID NO:23, wherein position 46 is not Glu; Asp, Gln or Asn and position 50 is not Ile, Leu, Ala, Gly or Val. In a still further preferred embodiment, the IL-15 mutein comprises or consists of an amino acid sequence of SEQ ID NO:33.

In yet another preferred embodiment, the at least one antigen is an IL-15 fragment, wherein said IL-15 fragment comprises or alternatively consists of at least one antigenic site.

It is known that possession of immunogenicity does not usually require the full length of a protein and usually a protein contains more than one antigenic epitope, i.e. antigenic site. A fragment or a short peptide may be sufficient to contain at least one antigenic site that can be bound immunospecifically by an antibody or by a T-cell receptor within the context of an MHC molecule. Antigenic site or sites can be determined by a number of techniques generally known to the skilled person in the art. It can be done by sequence alignment and structure prediction. By way of example, one can predict possible α-helices, turns, inter- and intra-chain disulfide bonds, etc. using a program such as Rasmol. One can further predict sequences that are buried within the molecule or sequences that are exposed on the surface of the molecule. Sequences exposed on the surface of the molecule are more likely to comprise natural antigenic site(s), and thus are useful in inducing therapeutic antibodies. After a surface peptide sequence has been determined, the antigenic site within this sequence can be further defined by, for example, exhaustive mutagenesis method (such as alanine scanning mutagenesis, Cunningham B C, Wells J A. Science 1989 Jun. 2; 244 (4908):1081-5). Briefly amino acids within this sequence are mutated to alanine one by one and the amino acids whose alanine mutations show respectively reduced binding to an antibody (raised against the wild type sequence) or lose totally the binding are likely component of the antigenic site.

Another method of determining antigenic site(s) is to generate overlapping peptides that covers the full-length sequence of IL-15 (Geysen, PNAS Vol 81: 3998-4002, (1984) and Slootstra, J. W. et al., (1996) Mol. Divers. 1, 87-96). Usually as initial screening, peptides of 20-30 amino acids in length with 5-10 amino acids overlap can be chemically synthesized. Mice are immunized with each individual peptide and polyclonal sera are taken from these mice. Whether the polyclonal sera recognize the native IL-15 protein can be tested using various methods such as ELISA or immunoprecipitation. Peptides, of which corresponding serum recognizes IL-15 protein contains most likely natural antigenic sites.

Peptide, when used alone as an antigen or linked to a carrier, may adapt a configuration that is different from that when it is in the context of the full length protein. Therefore, binding of peptide to polyclonal sera, obtained from mouse immunized with IL-15 shall be cross-checked.

Alternatively, a rodent is immunized with full length IL-15 protein. The cross reactivity of the resulted polyclonal serum with each individual, partially overlapping peptides are tested by a number of methods such as ELISA, immunoprecipitation or mass spectrometry. (Parker and Tomer, Mol. Biotechnol. 2002, 20, 49-62). These peptides can be of synthetic or recombinant origin.

Technologies to simplify and to facilitate the above mentioned procedures are available. For instance the peptides can be generated randomly and displayed on the surface of phage. (Nilsson, Methods Enzymol. 2000;326:480-505; Winter Annu Rev Immunol. 1994;12:433-55; peptide phage display, Smith, Methods Enzymol. 1993;217:228-57). The amount of partially overlapping peptides needed can be significantly reduced using the SPOT technology (Jerini S technology; Sigma-Genosys).

In a further preferred embodiment of the present invention, the IL-15 fragment comprises, or alternatively or preferably consists of, at least 5 to 12 contiguous amino of an IL-15 protein or an IL-15 mutein as defined herein.

In one preferred embodiment, the IL-15 fragment consists of less than 60, preferably less than 50, more preferably less than 40, even more preferably less than 30, still more preferably less than 20 amino acids in length.

In a further preferred embodiment, the IL-15 fragment comprises amino acid 44-52, preferably amino acid 44-54, more preferably amino acid 43-55 of SEQ ID NO:23. In one still further preferred embodiment, the IL-15 fragment has an amino acid sequence wherein position 46 of SEQ ID NO:23 is not Glu, preferably not Glu; Asp, Gln or Asn. In one alternative still further preferred embodiment, the IL-15 fragment has an amino acid sequence wherein position 50 of SEQ ID NO:23 is not Ile, preferably not Ile, Leu, Ala, Gly or Val.

In a further preferred embodiment, the IL-15 fragment comprises amino acid 64-68, preferably 62-70, more preferably 61-73 of SEQ ID NO:23.

In a preferred embodiment, the IL-15 fragment comprises or consists of an amino acid sequence selected from a group consisting of: (a) SEQ ID NO:34; (b) SEQ ID NO:35; (c) SEQ ID NO:36; (d) SEQ ID NO:37; (e) SEQ ID NO:38; (f) SEQ ID NO:39; (g) SEQ ID NO:40; and (h) an amino acid sequence which is at least 65%, preferably at least 80%, or more preferably at least 85%, even more preferably at least 90%, or most preferably at least 95% identical with any of SEQ ID NO:34-40.

The present invention provides for a method of producing the composition of the invention comprising (a) providing a VLP with at least one first attachment site; (b) providing at least one antigen, wherein said antigen is an IL-15 protein, an IL-15 mutein or an IL-15 fragment, with at least one second attachment site; and (c) combining said VLP and said at least one antigen to produce said composition, wherein said at least one antigen and said VLP are linked through the first and the second attachment sites. In a preferred embodiment, the provision of the at least one antigen, i.e. an IL-15 protein, an IL-15 mutein or an IL-15 fragment, with the at least one second attachment site is by way of expression, preferably by way of expression in a bacterial system, preferably in E. coli. Usually tag, such as His tag, Myc tag is added to facilitate the purification process. In another approach particularly the IL-15 fragments with no longer than 50 amino acids can be chemically synthesized.

In one preferred embodiment of the invention, the VLP with at least one first attachment site is linked to the IL-15 of the invention with at least one second attachment site via at least one peptide bond. Gene encoding IL-15 of the invention, preferably IL-15 fragment, more preferably a fragment not longer than 50 amino acids, even more preferably less than 30 amino acids, is in-frame ligated, either internally or preferably to the N- or the C-terminus to the gene encoding the coat protein of the VLP. Fusion may also be effected by inserting sequences of the IL-15 fragment into a mutant of a coat protein where part of the coat protein sequence has been deleted, that are further referred to as truncation mutants. Truncation mutants may have N- or C-terminal, or internal deletions of part of the sequence of the coat protein. For example for the specific VLP HBcAg, amino acids 79-80 are replaced with a foreign epitope. The fusion protein shall preferably retain the ability of assembly into a VLP upon expression which can be examined by electromicroscopy.

Flanking amino acid residues may be added to increase the distance between the coat protein and foreign epitope. Glycine and serine residues are particularly favored amino acids to be used in the flanking sequences. Such a flanking sequence confers additional flexibility, which may diminish the potential destabilizing effect of fusing a foreign sequence into the sequence of a VLP subunit and diminish the interference with the assembly by the presence of the foreign epitope.

In other embodiments, the at least one IL-15 of the invention, preferably the IL-15 fragment consisting of less than 50 amino acids can be fused to a number of other viral coat protein, as way of examples, to the C-terminus of a truncated form of the Al protein of Qβ (Kozlovska, T. M., et al., Intervirology 39:9-15 (1996)), or being inserted between position 72 and 73 of the CP extension. As another example, the IL-15 fragment can be inserted between amino acid 2 and 3 of the fr CP, leading to a IL-15-fr CP fusion protein (Pushko P. et al., Prot. Eng. 6:883-891 (1993)). Furthermore, IL-15 fragment can be fused to the N-terminal protuberant β-hairpin of the coat protein of RNA phage MS-2 (WO 92/13081). Alternatively, the IL-15 fragments can be fused to a capsid protein of papillomavirus, preferably to the major capsid protein L1 of bovine papillomavirus type 1 (BPV-1) (Chackerian, B. et al., Proc. Natl. Acad. Sci.USA 96:2373-2378 (1999), WO 00/23955). Substitution of amino acids 130-136 of BPV-1 L1 with an IL-15 fragment is also an embodiment of the invention. Further embodiments o fusing antigen of the invention to coat protein, mutants or fragements thereof, to a coat protein of a virus have been disclosed in WO 2004/009124 page 62 line 20 to page 68 line 17 and herein are incorporated by way of reference.

In another preferred embodiment, IL-15 of the invention, preferably IL-15 fragments, even more preferably IL-15 fragment with amino acid sequenced SEQ ID NO:34, 35, 36, 37, 38, 39 or 40 is fused to either the N- or the C-terminus of a coat protein, mutants or fragments thereof, of RNA phage AP205. In one further preferred embodiment, the fusion protein further comprises a spacer, wherein said spacer is positioned between the coat protein, fragments or mutants thereof, of AP205 and the IL-15 of the invention.

In one preferred embodiment of the present invention, the composition comprises or alternatively consists essentially of a virus-like particle with at least one first attachment site linked to at least one IL-15 of the invention with at least one second attachment site via at least one covalent bond, preferably the covalent bond is a non-peptide bond. In a preferred embodiment of the present invention, the first attachment site comprises, or preferably is, an amino group, preferably the amino group of a lysine residue. In another preferred embodiment of the present invention, the second attachment site comprises, or preferably is, a sulfhydryl group, preferably a sulfhydryl group of a cysteine.

In a very preferred embodiment of the invention, the at least one first attachment site comprises or preferably is an amino group, preferably an amino group of a lysine residue and the at least one second attachment site comprises or preferably is a sulfhydryl group, preferably a sulfhydryl group of a cysteine.

In one preferred embodiment of the invention, the IL-15 of the invention is linked to the VLP by way of chemical cross-linking, typically and preferably by using a heterobifunctional cross-linker. In preferred embodiments, the hetero-bifunctional cross-linker contains a functional group which can react with the preferred first attachment sites, preferably with the amino group, more preferably with the amino groups of lysine residue(s) of the VLP, and a further functional group which can react with the preferred second attachment site, i.e. a sulfhydryl group, preferably of cysteine(s) residue inherent of, or artificially added to the IL-15 of the invention, and optionally also made available for reaction by reduction. Several hetero-bifunctional cross-linkers are known to the art. These include the preferred cross-linkers SMPH (Pierce), Sulfo-MBS, Sulfo-EMCS, Sulfo-GMBS, Sulfo-SIAB, Sulfo-SMPB, Sulfo-SMCC, SVSB, SIA and other cross-linkers available for example from the Pierce Chemical Company, and having one functional group reactive towards amino groups and one functional group reactive towards sulfhydryl groups. The above mentioned cross-linkers all lead to formation of an amide bond after reaction with the amino group and a thioether linkage with the sulfhydryl groups. Another class of cross-linkers suitable in the practice of the invention is characterized by the introduction of a disulfide linkage between the IL-15 of the invention and the VLP upon coupling. Preferred cross-linkers belonging to this class include, for example, SPDP and Sulfo-LC-SPDP (Pierce).

In a preferred embodiment, the composition of the invention further comprises a linker. Engineering of a second attachment site onto the IL-15 of the invention is achieved by the association of a linker, preferably containing at least one amino acid suitable as second attachment site according to the disclosures of this invention. Therefore, in a preferred embodiment of the present invention, a linker is associated to the IL-15 of the invention by way of at least one covalent bond, preferably, by at least one, typically one peptide bond. Preferably, the linker comprises, or alternatively consists of, the second attachment site. In a further preferred embodiment, the linker comprises a sulfhydryl group, preferably of a cysteine residue. In another preferred embodiment, the amino acid linker is a cysteine residue.

The selection of a linker will be dependent on the nature of the IL-15 of the invention, on its biochemical properties, such as pI, charge distribution and glycosylation. In general, flexible amino acid linkers are favored. In a further preferred embodiment of the present invention, the linker consists of amino acids, wherein further preferably the linker consists of at most 25, preferably at most 20, more preferably at most 15 amino acids. In an again preferred embodiment of the invention, the amino acid linker contains no more than 10 amino acids. Preferred embodiments of the linker are selected from the group consisting of: (a) CGG or CG/GC; (b) N-terminal gamma 1-linker (e.g. CGDKTHTSPP, SEQ ID NO:44); (c) N-terminal gamma 3-linker (e.g. CGGPKPSTPPGSSGGAP, SEQ ID NO:55); (d) Ig hinge regions; (e) N-terminal glycine linkers (e.g. GCGGGG, SEQ ID NO:45); (f) (G)kC(G)n with n=0-12 and k=0-5; (g) N-terminal glycine-serine linkers ((GGGGS)n, n=1-3 with one further cysteine (for example SEQ ID NO:46, which corresponds to an embodiment wherein n=1); (h) (G)kC(G)m(S)l(GGGGS)n with n=0-3, k=0-5, m=0-10, l=0-2 (for example SEQ ID NO:47, which corresponds to an embodiment wherein n=1, k=1, l=1 and m=1); (i) GGC; (k) GGC-NH2; (l) C-terminal gamma 1-linker (e.g. DKTHTSPPCG, SEQ ID NO:48); (m) C-terminal gamma 3-linker (e.g. PKPSTPPGSSGGAPGGCG, SEQ ID NO:49); (n) C-terminal glycine linkers (GGGGCG, SEQ ID NO:50); (o) (G)nC(G)k with n=0-12 and k=0-5; (p) C-terminal glycine-serine linkers ((SGGGG)n n=1-3 with one further cysteine (for example SEQ ID NO:51, which corresponds to an embodiment wherein n=1); (q) (G)m(S)l(GGGGS)n(G)oC(G)k with n=0-3, k=0-5, m=0-10, l=0-2, and o=0-8 (for example SEQ ID NO:52, which corresponds to an embodiment wherein n=1, k=1, l=1, o=1 and m=1). In a further preferred embodiment the linker is added to the N-terminus of IL-15 of the invention. In another preferred embodiment of the invention, the linker is added to the C-terminus of IL-15 of the invention.

Preferred linkers according to this invention are glycine linkers (G)n further containing a cysteine residue as second attachment site, such as N-terminal glycine linker (GCGGGG) and C-terminal glycine linker (GGGGCG). Further preferred embodiments are C-terminal glycine-lysine linker (GGKKGC, SEQ ID NO:53) and N-terminal glycine-lysine linker (CGKKGG, SEQ ID NO:54), GGCG a GGC or GGC-NH2 (“NH2” stands for amidation) linkers at the C-terminus of the peptide or CGG at its N-terminus. In general, glycine residues will be inserted between bulky amino acids and the cysteine to be used as second attachment site, to avoid potential steric hindrance of the bulkier amino acid in the coupling reaction.

Linking of the IL-15 of the invention to the VLP by using a hetero-bifunctional cross-linker according to the preferred methods described above, allows coupling of the IL-15 of the invention to the VLP in an oriented fashion. Other methods of linking the IL-15 of the invention to the VLP include methods wherein the IL-15 of the invention is cross-linked to the VLP, using the carbodiimide EDC, and NHS. The IL-15 of the invention may also be first thiolated through reaction, for example with SATA, SATP or iminothiolane. The IL-15 of the invention, after deprotection if required, may then be coupled to the VLP as follows. After separation of the excess thiolation reagent, the IL-15 of the invention is reacted with the VLP, previously activated with a hetero-bifunctional cross-linker comprising a cysteine reactive moiety, and therefore displaying at least one or several functional groups reactive towards cysteine residues, to which the thiolated IL-15 of the invention can react, such as described above. Optionally, low amounts of a reducing agent are included in the reaction mixture. In further methods, the IL-15 of the invention is attached to the VLP, using a homo-bifunctional cross-linker such as glutaraldehyde, DSG, BM[PEO]4, BS3, (Pierce) or other known homo-bifunctional cross-linkers with functional groups reactive towards amine groups or carboxyl groups of the VLP.

In other embodiments of the present invention, the composition comprises or alternatively consists essentially of a virus-like particle linked to IL-15 of the invention via chemical interactions, wherein at least one of these interactions is not a covalent bond. For example, linking of the VLP to the IL-15 of the invention can be effected by biotinylating the VLP and expressing the IL-15 of the invention as a streptavidin-fusion protein. Other binding pairs, such as ligand-receptor, antigen-antibody, can also be used as coupling reagent in a similar manner as biotin-avidin.

U.S. Pat. No. 5,698,424 describes a modified coat protein of bacteriophage MS-2 capable of forming a capsid, wherein the coat protein is modified by an insertion of a cysteine residue into the N-terminal hairpin region, and by replacement of each of the cysteine residues located external to the N-terminal hairpin region by a non-cysteine amino acid residue. The inserted cysteine may then be linked directly to a desired molecular species to be presented such as an epitope or an antigenic protein.

We note, however, that the presence of an exposed free cysteine residue in the capsid may lead to oligomerization of capsids by way of disulfide bridge formation. Moreover, attachment between capsids and antigenic proteins by way of disulfide bonds are labile, in particular, to sulfhydryl-moiety containing molecules, and are, furthermore, less stable in serum than, for example, thioether attachments (Martin F J. and Papahadjopoulos D.(1982) Irreversible Coupling of Immunoglobulin Fragments to Preformed Vesicles. J. Biol. Chem. 257: 286-288).

Therefore, in a further very preferred embodiment, the linkage of the VLP and the at least one antigen does not comprise a disulfide bond. Further preferred hereby, the at least one second attachment comprise, or preferably is, a sulfhydryl group. Moreover, in again a very preferred embodiment of the invention, the linkage of the VLP and the at least one antigen does not comprise a sulphur-sulphur bond. In a further very preferred embodiment, said at least one first attachment site is not or does not comprise a sulfhydryl group of a cysteine. In again a further very preferred embodiment, said at least one first attachment site is not or does not comprise a sulfhydryl group.

In one preferred embodiment of the invention, the VLP is recombinantly produced in a host, and wherein the VLP is essentially free of host RNA, preferably host nucleic acids or wherein the VLP is essentially free of host DNA, preferably host nucleic acids. In one preferred embodiment, the VLP of an RNA phage is recombinantly produced in a host, and wherein the VLP of an RNA phage is essentially free of host RNA, preferably host nucleic acids.

In one further preferred embodiment, the composition further comprises at least one polyanionic macromolecule bound to, preferably packaged inside or enclosed in, the VLP. In a still further preferred embodiment, the polyanionic macromolecule is polyglutamic acid and/or polyaspartic acid. In one preferred embodiment, the VLP is of an RNA phage. Reducing or eliminating the amount of host RNA, preferably host nucleic acids, minimizes or reduces unwanted T cell responses, such as inflammatory T cell responses and cytotoxic T cell responses, and other unwanted side effects, such as fever, while maintaining strong antibody response specifically against IL-15.

Essentially free of host RNA (or DNA), preferably host nucleic acids: The term “essentially free of host RNA (or DNA), preferably host nucleic acids” as used herein, refers to the amount of host RNA (or DNA), preferably host nucleic acids, comprised by the VLP, which is typically and preferably less than 30 pg, preferably less than 20 pg, more preferably less than 10 pg, even more preferably less than 8 pg, even more preferably less than 6 pg, even more preferably less than 4 pg, most preferably less than 2 pg, per mg of the VLP. Host, as used within the afore-mentioned context, refers to the host in which the VLP is recombinantly produced. Conventional methods of determining the amount of RNA (or DNA), preferably nucleic acids, are known to the skilled person in the art. The typical and preferred method to determine the amount of RNA, preferably nucleic acids, in accordance with the present invention is described in Example 17 of the PCT/EP2005/055009 filed on Oct. 5, 2005 by the same assignee. Identical, similar or analogous conditions are, typically and preferably, used for the determination of the amount of RNA (or DNA), preferably nucleic acids, for inventive compositions comprising VLPs other than Qβ. The modifications of the conditions eventually needed are within the knowledge of the skilled person in the art.

The term “polyanionic macromolecule”, as used herein, refers to a molecule of high relative molecular mass which comprises repetitive groups of negative charge, the structure of which essentially comprises the multiple repetitions of units derived, actually or conceptually, from molecules of low relative molecular mass.

In one aspect, the invention provides a vaccine comprising the composition of the invention. In one preferred embodiment, the IL-15 of the invention linked to the VLP in the vaccine composition may be of animal, preferably mammal or human origin. In preferred embodiments, the IL-15 of the invention is of human, bovine, dog, cat, mouse, rat, pig or horse origin.

In one preferred embodiment, the vaccine composition further comprises at least one adjuvant. The administration of the at least one adjuvant may hereby occur prior to, contemporaneously or after the administration of the inventive composition. The term “adjuvant” as used herein refers to non-specific stimulators of the immune response or substances that allow generation of a depot in the host which when combined with the vaccine and pharmaceutical composition, respectively, of the present invention may provide for an even more enhanced immune response.

In another preferred embodiment, the vaccine composition is devoid of adjuvant. An advantageous feature of the present invention is the high immunogenicity of the composition, even in the absence of adjuvants. The absence of an adjuvant, furthermore, minimizes the occurrence of unwanted inflammatory T-cell responses representing a safety concern in the vaccination against self antigens. Thus, the administration of the vaccine of the invention to a patient will preferably occur without administering at least one adjuvant to the same patient prior to, contemporaneously or after the administration of the vaccine.

The invention further discloses a method of immunization comprising administering the vaccine of the present invention to an animal or a human. The animal is preferably a mammal, such as cat, sheep, pig, horse, bovine, dog, rat, mouse and particularly human. The vaccine may be administered to an animal or a human by various methods known in the art, but will normally be administered by injection, infusion, inhalation, oral administration, or other suitable physical methods. The conjugates may alternatively be administered intramuscularly, intravenously, transmucosally, transdermally, intranasally, intraperitoneally or subcutaneously. Components of conjugates for administration include sterile aqueous (e.g., physiological saline) or non-aqueous solutions and suspensions. Examples of non-aqueous solvents are propylene glycol, polyethylene glycol, vegetable oils such as olive oil, and injectable organic esters such as ethyl oleate. Carriers or occlusive dressings can be used to increase skin permeability and enhance antigen absorption.

Vaccines of the invention are said to be “pharmacologically acceptable” if their administration can be tolerated by a recipient individual. Further, the vaccines of the invention will be administered in a “therapeutically effective amount” (i.e., an amount that produces a desired physiological effect). The nature or type of immune response is not a limiting factor of this disclosure. Without the intention to limit the present invention by the following mechanistic explanation, the inventive vaccine might induce antibodies which bind to IL-15 and thus reducing its concentration and/or interfering with its physiological or pathological function.

In one aspect, the invention provides a pharmaceutical composition comprising the composition as taught in the present invention and an acceptable pharmaceutical carrier. When vaccine of the invention is administered to an individual, it may be in a form which contains salts, buffers, adjuvants, or other substances which are desirable for improving the efficacy of the conjugate. Examples of materials suitable for use in preparation of pharmaceutical compositions are provided in numerous sources including REMINGTON'S PHARMACEUTICAL SCIENCES (Osol, A, ed., Mack Publishing Co., (1990)).

The invention teaches a process for producing the composition of the invention comprising the steps of: (a) providing a VLP with at least one first attachment site; (b) providing a IL-15 of the invention with at least one second attachment site, and (c) combining said VLP and said IL-15 of the invention to produce a composition, wherein said IL-15 of the invention and said VLP are linked through the first and the second attachment sites.

In a further preferred embodiment, the step of providing a VLP with at least one first attachment site comprises further steps: (a) disassembling said virus-like particle to said coat proteins, mutants or fragments thereof, of said RNA-bacteriophage; (b) purifying said coat proteins, mutants or fragments thereof; (c) reassembling said purified coat proteins, mutants or fragments thereof, of said RNA-bacteriophage to a virus-like particle, wherein said virus-like particle is essentially free of host RNA, preferably host nucleic acids. In a still further preferred embodiment, the reassembling of said purified coat proteins is effected in the presence of at least one polyanionic macromolecule.

The invention provides a method for treating and/or attenuating diseases or conditions in which IL-15 exerts an important pathological function in an animal or in human, wherein said method comprises administering the inventive composition of the invention to an animal or to a human suffering from said disease or said condition. In a preferred embodiment, said disease or condition in which IL-15 exerts an important pathological function is selected from the group consisting of atherosclerosis, asthma, transplant rejection and inflammatory and/or chronic autoimmune diseases, for example but not limited to, rheumatoid arthritis, psoriatic arthritis, juvenile idiopathic arthritis, psoriasis. Alternatively the invention provides a use of the inventive composition for the manufacture of a medicament for treatment of a disease selected from the group consisting of atherosclerosis, asthma, transplantation rejection and an inflammatory and/or chronic autoimmune disease in an animal or preferably in a human.

In one aspect, the invention provides a method of treating a disease in an animal or a human comprising administering at least one IL-15 antagonist to said animal or human, wherein said disease is selected from a group consisting of atherosclerosis and asthma. Alternatively the invention provides a use of at least one IL-15 antagonist for the manufacture of a medicament for treatment of a disease selected from the group consisting of atherosclerosis and asthma.

An “IL-15 antagonist” inhibits IL-15 function by various means, such as, but not limited to, (i) decreasing the IL-15 concentration in the blood, (ii) preventing IL-15 from binding to IL-15 receptor complex, preferably preventing IL-15 from binding to the a subunit of the IL-15 receptor complex, or (iii) preventing IL-15 from transducing a signal to a cell through either the β or the γ subunits of the IL-15 receptor complex, thereby by antagonizing IL-15 biological activity. Typically and preferably the binding of IL-15 to the IL-15 receptor complex, preferably to the a subunit can be checked by in vitro binding assays, for example as described in J. Biol Chem. 2004 Jun. 4;279(23):24313-22. Typically and preferably the IL-15 function, typically and preferably its function for stimulation of T-cell proliferation, can be checked by in intro assays, for example, as described in EXAMPLE 2 in EP 0772624.

In one preferred embodiment, the IL-15 antagonist is an antibody specifically binding to IL-15. The binding of an antibody to IL-15 may result in the clearance of the formed antigen-antibody complex and thereby decrease the IL-15 concentration in the blood. Furthermore, the binding of an antibody to IL-15 may prevent the binding of IL-15 to its receptor and thus prevents IL-15 from exerting its activity through its receptor. In addition the binding of an antibody to IL-15 may not interfere the binding of IL-15 to its receptor, however, the presence of antibody may prevent the signal transduction mediated by the β or the y subunits of the IL-15 receptor complex.

IL-15 antibody could be polyclonal or monoclonal and could be generated by immunization of different animal species, such as mouse, rat, rabbit or human. Monoclonal antibody, depending on the techniques used, may be a murine, a chimeric, a CDR-grafted, a humanized, a human or a synthesized antibody. Thus the term “monoclonal antibody” means an antibody composition having a homogeneous antibody population. It is not intended to be limited as regards to the source of the antibody or the manner in which it is made. In one preferred embodiment, said IL-15 antagonist comprises or is a functional fragment of said antibody. Monoclonal antibodies specifically bind to IL-15 are available in the art.

In one preferred embodiment, said IL-15 antagonist is a monoclonal antibody with a binding affinity (Ka) of 10⁷ M⁻¹ or greater, preferably 10⁸ M⁻¹ or greater, and more preferably 10⁹ M⁻¹ or greater.

In one preferred embodiment, said IL-15 antagonist is a monoclonal antibody which inhibits IL-15 induced T-cell proliferation with an IC50 value of less than 100 nM, preferably less than 10 nM as determined by proliferation inhibition assay, which typically and preferably can be carried as described in EXAMPLE 8 of WO03/017935.

In one preferred embodiment, said IL-15 antagonist is a monoclonal antibody HuMax-IL-15 (also named 146B7, AMG714) or a fragment thereof, as described in J Clin Invest 2003, 112, 1571, in Arthritis & Rheumatism. 2005, 52, 2686 and in WO 03/017935.

In one preferred embodiment, said IL-15 antagonist is a monoclonal antibody obtained from the hybridoma selected from the group consisting of: (i) ATCC accession number M110; (ii) ATCC accession number M111; (iii) ATCC accession number M112, ((i)-(iii) can be referenced to WO 9626274); and (iv) 146H5 (iv) can be referenced to WO03/017935.

In one preferred embodiment, said IL-15 antagonist is an antibody specifically binding to IL-15 and wherein preferably said antibody is produced in response to the inventive composition of the invention. Preferably said antibody is generated in the body of an animal or a human, who has received the inventive composition or the inventive vaccine, preferably according to the inventive immunization method of the invention. In one preferred embodiment, the antibody is a monoclonal antibody generated by immunizing mouse of the inventive composition of the invention. Preferably so generated antibody will be further modified or engineered for the optimization of human use using available techniques to date.

In one preferred embodiment, said IL-15 antagonist comprises or is an IL-15 soluble receptor, or a fragment thereof. In one preferred embodiment, said IL-15 antagonist comprises or is an IL-15 soluble receptor a subunit, or a fragment thereof. In one preferred embodiment, said IL-15 antagonist comprises or is the extracellular domain of IL-15 receptor a subunit, or a fragment thereof. In one further preferably embodiment, said IL-15 antagonist comprises or consists of the amino acid sequence as set forth in SEQ ID NO:41 or an amino acid sequence which has at least 80%, preferably 85%, more preferably 90%, more preferably 95%, more preferably 97% identity to SEQ ID NO:41.

In one preferred embodiment, said IL-15 antagonist comprises or is an IL-15 mutein. In one further preferred embodiment, said IL-15 mutein is still capable of binding to IL-15 receptor a subunit and prevents IL-15 from transducing a signal to the cells through either the β or the γ subunits. In one preferred embodiment, said IL-15 mutein comprises or consists of an amino acid sequence as set forth in SEQ ID NO:23, wherein at least one position, preferably two, more preferably all three positions of Asp8, GIn101, and Gln108 of SEQ ID NO:23 is/are mutated, preferably substituted, preferably by non-conservative substitution. In one preferred embodiment, said IL-15 mutein comprises or consists of an amino acid sequence as set forth in SEQ ID NO:23, wherein at least one or both Gln101 and Gln108 are deleted or preferably substituted. In one further preferred embodiment, said IL-15 mutein comprises or consists of an amino acid sequence as set forth in SEQ ID NO:42.

In one preferred embodiment, said IL-15 mutein comprises or consists of an amino acid sequence as set forth in SEQ ID NO:23, wherein at least one, or preferably both Asp8 and GIn108 are deleted or preferably substituted, preferably with a different naturally occurring amino acid residue, further preferably with a serine or a cysteine. In one alternatively preferred embodiment Gln108 is substituted to Asp. In one alternatively preferred embodiment, Asp8 is substituted to Arg or to Lys.

In one preferred embodiment, said IL-15 mutein comprises or consists of an amino acid sequence which is at least 80%, preferably at least 85%, more preferably at least 90%, or most preferably at least 95% identical with SEQ ID NO:23 and wherein at least one position, preferably two, more preferably all three positions corresponding to Asp8, GIn101, and Gln108 of SEQ ID NO:23 is/are mutated, preferably substituted, preferably by non-conservative substitution. In one preferred embodiment, said IL-15 mutein comprises or consists of an amino acid sequence which is at least 80%, preferably at least 85%, more preferably at least 90%, or most preferably at least 95% identical with SEQ ID NO:42, wherein the position corresponding to 101 and 108 of SEQ ID NO:42 remain Asp.

### EXAMPLES

Qβ VLPs, AP205 VLPs and the like, as used within this example section, refer to VLPs obtained by recombinant expression from E. coli and subsequent purification as described in WO 02/056905, WO 04/007538.

### Example 1

### Construction of pM-IL-15-FL-CG

The sequence from BamHI site to PmeI site of the plasmid pModEC1 (WO 03/040164 A2) was changed to catatggatc cgctagccct cgagga ctac aaggatgacg acgacaaggg tggttgcggt taataagttt aaacgcggcc gc (SEQ ID NO:43) by replacing the original with annealed oligos B-FL-L-P R (SEQ ID NO:34) and B-FL-C-P F (SEQ ID NO:35). The resulting construct was termed pMod-FL-CG, which had a Nde I, BamH I, Nhel, XhoI, Pmel and NotI restriction sites in its multiple cloning sites.

Mouse IL-15 was amplified from a cDNA library of activated dendritic cell by PCR using the following primers: IL-15-F (SEQ ID NO:36) and IL-15 -Xho-R (SEQ ID NO:37). IL-15-F had an internal Ndel site and IL-15-XhoI had an internal XhoI site. The PCR product was digested with Ndel and XhoI and ligated into pMod-FL-CG digested with the same enzymes. The resulting plasmid was named pM-IL-15-FC-CG, which encodes a fusion protein comprising mouse IL-15, a flag tag and a linker containing cysteine at the C-terminus (SEQ ID NO:30).

### Example 2

### Expression of pM-IL-15-FL-CG

Competent E. coli BL21 (DE3) cells were transformed with plasmid pM-IL-15-FL-CG. Single colonie from ampicillin (Amp)-containing agar plates was expanded in liquid culture (SB with 150 mM MOPS, pH 7.0, 100 μg/ml Amp) and incubated at 30° C. with 220 rpm shaking overnight. Overnight culture was then diluted 1:50 into the same medium and grew to OD600=2.8 at 30° C. Expression was induced with 1 mM IPTG. Cells were harvested after 4 hours' induction by centrifuging at 6000 rpm for 10 minutes. Cell pellet was suspended in lysis buffer (10 mM Na₂HPO₄, 30 mM NaCl, 10 mM EDTA and 0.25% Tween-20) with 0.8 mg/ml lysozyme, sonicated and treated with benzonase. After contrifugation with 48000 RCF for 20 minutes, the supernatant was resolved in 12% PAGE gel and the mouse IL-15 expression was confirmed by anti-mouse IL-15 (R&D system) on Western blot, which clearly demonstrated the expression of IL-15-FL-CG which run at the expected molecular weight of 14.9 KD.

### Example 3

### Purification of IL-15-FL-CG

IL-15-FL-CG was first purified via an anti-FLAG M2 column. Briefly, IL-15-FL-CG lysate was loaded on the anti-FLAG M2 column. Unbound contaminants were washed away with TBS (50 mM Tris HCl, 150 mM NaCl, pH 7.4). IL-15-FL-CG was then eluted from the column with FLAG peptide (100 μg/ml). The elute was further purified by Q Fast Flow column.

### Example 4

### Production of Human IL-15 Protein, IL-15 Muteins and IL-15 Fragments

Human IL-15 (SEQ ID NO:23) is amplified from a cDNA library of activated dendritic cell by PCR using substantially the same protocol as described in EXAMPLE 1 and the PCR product is ligated into pMod-FL-CG. The resulting plasmid is named pH-IL-15-FC-CG, which encodes a fusion protein comprising human IL-15, a flag tag and a linker containing cysteine at the C-terminus.

Substantially the same protocol as described in EXAMPLE 1 is used to construct plasmid expressing human IL-15 muteins (SEQ ID NO:31, 32, or 33). Substantially the same protocols as described in EXAMPLE 2 and 3 are applied to express and purify human IL-15 protein, human IL-15 muteins.

Various IL-15 fragments (SEQ ID NO:34-40) are chemically synthesized according to standard protocols. An additional cysteine is fused to the N-terminus of each of the sequence of IL-15 fragments

### Example 5

### Preparation of Qβ VLPs of the Invention by Disassembly/Reassembly in the Presence of Different Polyanionic Macromolecules Resulting in Reassembled Qβ VLPs

(A) Disassembly of Qβ VLP

45 mg Qβ VLP (2.5 mg/ml, as determined by Bradford analysis) in PBS (20 mM Phosphate, 150 mM NaCl, pH 7.5) purified from E. coli lysate was reduced with 10 mM DTT for 15 min at room temperature under stirring conditions. Magnesium chloride was then added to 0.7 M final concentration and the incubation was continued for 15 min at room temperature under stirring conditions, which led to the precipitation of the encapsulated host cell RNA. The solution was centrifuged for 10 min at 4000 rpm at 4° C. (Eppendorf 5810 R, in fixed angle rotor A-4-62 used in all following steps) in order to remove the precipitated RNA from the solution. The supernatant, containing the released, dimeric Qβ coat protein, was used for the chromatographic purification steps.

(B) Purification of the Qβ coat protein by cation exchange chromatography and by size exclusion chromatography

The supernatant of the disassembly reaction, containing the dimeric coat protein, host cell proteins and residual host cell RNA, was diluted 1:15 in water to adjust conductivity below 10 mS/cm and was loaded onto a SP-Sepharose FF column (xk16/20, 6 ml, Amersham Bioscience). The column was equilibrated beforehand with 20 mM sodium phosphate buffer pH 7. The elution of the bound coat protein was accomplished by a step gradient to 20 mM sodium phosphate/500 mM sodium chloride and the protein was collected in a fraction volume of approx. 25 ml. The chromatography was carried out at room temperature with a flow rate of 5 mi/min and the absorbance was monitored at 260 nm and 280° nm.

In the second step, the isolated Qβ coat protein (the eluted fraction from the cation exchange column) was loaded (in two runs) onto a Sephacryl S-100 HR column (xk26/60, 320 ml, Amersham Bioscience), equilibrated with 20 mM sodium phosphate/250 mM sodium chloride; pH 6.5. The chromatography was carried out at room temperature with a flow rate of 2.5 mi/min and the absorbance was monitored at 260 nm and 280 nm. Fractions of 5 ml were collected.

(C1) Reassembly of the Qβ VLP by dialysis

Purified Qβ coat protein (2.2 mg/ml in 20 mM sodium phosphate pH 6.5), one polyanionic macromolecule (2 mg/ml in water), urea (7.2 M in water) and DTT (0.5 M in water) were mixed to the final concentrations of 1.4 mg/ml coat protein, 0.14 mg/ml of the respective polyanionic macromolecule, 1 M urea and 2.5 mM DTT. The mixtures (1 ml each) were dialyzed for 2 days at 5° C. in 20 mM TrisHCl, 150 mM NaCl pH 8, using membranes with 3.5 kDa cut off. The polyanionic macromolecules were: polygalacturonic acid (25000-50000, Fluka), dextran sulfate (MW 5000 and 10000, Sigma), poly-L-aspartic acid (MW 11000 and 33400, Sigma), poly-L-glutamic acid (MW 3000, 13600 and 84600, Sigma) and tRNAs from bakers yeast and wheat germ.

(C2) Reassembly of the Qβ VLP by diafiltration

33 ml purified Qβ coat protein (1.5 mg/ml in 20 mM sodium phosphate pH 6.5, 250 mM NaCl) was mixed with water and urea (7.2 M in water), NaCl (5 M in water) and poly-L-glutamic acid (2 mg/ml in water, MW: 84600). The volume of the mixture was 50 ml and the final concentrations of the components were 1 mg/ml coat protein, 300 mM NaCl, 1.0 M urea and 0.2 mg/ml poly-L-glutamic acid. The mixture was then diafiltrated at room temperature, against 500 ml of 20 mM TrisHCl pH 8, 50 mM NaCl, applying a cross flow rate of 10 ml/min and a permeate flow rate of 2.5 ml/min, in a tangential flow filtration apparatus using a Pellicon XL membrane cartridge (Biomax 5K, Millipore).

### Example 6

### In Vitro Assembly of AP205 VLPs

(A) Purification of AP205 coat protein

Disassembly: 20 ml of AP205 VLP solution (1.6 mg/ml in PBS, purified from E.coli extract) was mixed with 0.2 ml of 0.5 M DTT and incubated for 30 min at room temperature. 5 ml of 5 M NaCl was added and the mixture was then incubated for 15 min at 60° C., causing precipitation of the DTT-reduced coat proteins. The turbid mixture was centrifuged (rotor Sorvall SS34, 10000 g, 10 min, 20° C.) and the supernatant was discarded and the pellet was dispersed in 20 ml of 1 M Urea/20 mM Na Citrate pH 3.2. After stirring for 30 min at room temperature, the dispersion was adjusted to pH 6.5 by addition of 1.5 M Na₂HPO₄ and then centrifuged (rotor Sorvall SS34, 10000 g, 10 min, 20° C.) to obtain supernatant containing dimeric coat protein.

Cation exchange chromatography: The supernatant (see above) was diluted with 20 ml water to adjust a conductivity of approx. 5 mS/cm. The resulting solution was loaded on a column of 6 ml SP Sepharose FF (Amersham Bioscience) which was previously equilibrated with 20 mM sodium phosphate pH 6.5 buffer. After loading, the column was washed with 48 ml of 20 mM sodium phosphate pH 6.5 buffer followed by elution of the bound coat protein by a linear gradient to 1 M NaCl over 20 column volumes. The fractions of the main peak were pooled and analyzed by SDS-PAGE and UV spectroscopy. According to SDS-PAGE, the isolated coat protein was essentially pure from other protein contaminations. According to the UV spectroscopy, the protein concentration was 0.6 mg/ml (total amount 12 mg), taking that 1 A280 unit reflects 1.01 mg/ml of AP205 coat protein. Furthermore, the value of A280 (0.5999) over the value of A260 (0.291) is 2, indicating that the preparation is essentially free of nucleic acids.

### (B) Assembly of AP205 VLPs

Assembly in the absence of any polyanionic macromolecule: The eluted protein fraction from above was diafiltrated and concentrated by TFF to a protein concentration of 1 mg/ml in 20 mM sodium phosphate pH 6.5. 500 pi of that solution was mixed with 50 pi of 5 M NaCl solution and incubated for 48 h at room temperature. The formation of reassembled VLPs in the mixture was shown by non-reducing SDS-PAGE and by size exclusion HPLC. A TSKgel G5000 PWXL column (Tosoh Bioscience), equilibrated with 20 mM sodium phosphate, 150 mM NaCl pH 7.2, was used for the HPLC analysis.

Assembly in the presence of polyglutamic acid: 375 μl of purified AP205 coat protein (1 mg/ml in 20 mM sodium phosphate pH 6.5) was mixed with 50 μl of NaCl stock solution (5 M in water) solution, 50 μl of polyglutamic acid stock solution (2 mg/ml in water, MW: 86400, Sigma) and 25 μl of water. The mixture was incubated for 48 h at room temperature. The formation of reassembled VLP in the mixture was shown by non-reducing SDS-PAGE and by size exclusion HPLC. The coat protein in the mixture was almost completely incorporated into the VLPs, showing a higher assembly efficiency than the AP205 coat protein assembled in the absence of any polyanionic macromolecule.

### Example 7

### Coupling IL-15-FL-CG to Qβ VLPs and Reassembled Qβ VLPs

Purified mouse IL-15-FL-CG (153 μM) obtained from EXAMPLE 3 was reduced for 1 hour with an equimolar TCEP in TBS pH 7.4. Reduced IL-15-FL-CG (83 μM) was incubated overnight at room temperature with 59 μM Qβ derivatized with SMPH in a total volume of 50 pt. The coupling reaction was analysed by SDS-PAGE and Western-Blot with anti-FLAG antibodies Protein concentration was measured by Bradford. The coupling efficiency was estimated, by densitometric analysis of the Coomassie blue stained SDS-PAGE.

Substantially the same experimental conditions are applied to couple human IL-15-FL-CG (obtained from EXAMPLE 4) to the reassembled Qβ VLP, which is obtained from Example 5 or the reassembled AP205 VLP, obtained from EXAMPLE 6.

### Example 8

### Coupling Human IL-15 Muteins to Qβ VLPs and the Reassembled Qβ VLP

Purified human IL-15 muteins (153 μM) obtained from EXAMPLE 4 are reduced for 1 hour with an equal molar TCEP in TBS pH 7.4. Reduced IL-15 muteins (83 μM) are incubated overnight at room temperature with 59 μM Qβ VLPs or 59 μM reassembled Qβ VLPs derivatized with SMPH in a total volume of 50 μl. The coupling reactions are analysed by SDS-PAGE and Western-Blot with anti-FLAG antibodies Protein concentrations are measured by Bradford. The coupling efficiency is estimated, by densitometric analysis of the Coomassie blue stained SDS-PAGE.

### Example 9

### Coupling Human IL-15 Protein to HBcAg1-185-Lys

Construction of HBcAg1-185-Lys, its expression and purification have been substantially described in EXAMPLE 2-5 of WO 03/040164. A solution of 120 μM HBcAg1-185-Lys capsid in 20 mM Hepes, 150 mM NaCl pH 7.2 is reacted for 30 minutes with a 25 fold molar excess of SMPH (Pierce), diluted from a stock solution in DMSO, at 25° C. on a rocking shaker. The reaction solution is subsequently dialyzed twice for 2 hours against 1 L of 20 mM Hepes, 150 mM NaCl, pH 7.2 at 4° C. The dialyzed HBcAg1-185-Lys reaction mixture is then reacted with the human IL-15 protein obtained in EXAMPLE 4. In the coupling reaction the human IL-15 protein is in twofold molar excess over the derivatized HBcAg1-185-Lys capsid. The coupling reaction proceeds for four hours at 25° C. on a rocking shaker. Coupling products are analysed by SDS-PAGE.

### Example 10

### Immunogenicity

In experiment A group of mice (n=5) were immunized with 50 μg Qβ VLPs coupled with mouse IL-15-FL-CG subcutaneously at day 0, day 14 and day 28 in the absence of any adjuvant. As negative controls, five mice were immunized with PBS only.

In experiment B group of mice (n=5) were immunized with 25 μg Qβ VLPs coupled with mouse IL-15-FL-CG subcutaneously at day 0, day 14 and day 28 in the absence of any adjuvant. As negative controls, five mice were immunized with Qβ VLPs only.

Table 1 demonstrates that immunization with Qβ-IL-15-FL-CG elicited high titers of IL-15 specific IgG antibodies in all mice as shown by ELISA. This demonstrates that the vaccine could overcome immunological tolerance to IL-15 without the addition of any adjuvant. The ELISA titer is defined as the serum dilution which results in half maximal optical density at 450 nm (OD 50%). ELISA plates were coated with recombinant IL-15. Averages of 5 animals are given with standard deviations.

Similar experimental conditions are applied to immunize mice with mouse IL-15-FL-CG coupled to the reassembled Qβ VLP, the antibody titer is measured by ELISA and compared with the antibody titer induced by IL-15-FL-CG coupled to Qβ VLPs and the negative controls.

TABLE 1
A (Experiment A)

| | Days after | | | | | |
|---|---|---|---|---|---|---|
| | immunizazion | | | | | |
| | d0 | d14 | d21 | d42 | d56 | d70 |
| Anti-IL-15 | 190 ± 253 | 2043 ± 3249 | 14487 ± 1212 | 72131 ± 39347 | 56772 ± 13403 | 32531 ± 15247 |
| antibody titer | | | | | | |

B (Experiment B)

| | Days after | | | | |
|---|---|---|---|---|---|
| | immunization | | | | |
| | d0 | d14 | d21 | d35 | d49 |
| Anti-IL-15 | 0 ± 0 | 6478 ± 9602 | 29294 ± 20111 | 53189 ± 58917 | 39551 ± 41976 |
| antibody titer | | | | | |

### Example 11

### Efficacy of Qβ VLP-IL-15 Vaccine in a Mouse Model of Rheumatoid Arthritis

The ability of the Qβ VLP-IL-15 vaccine to reduce arthritic symptoms in vivo was evaluated in a mouse model of Rheumatoid Arthritis (RA). In this model RA was induced by intravenous injection of a combination of 4 different monoclonal antibodies (Arthrogenic Monoclonal Antibody Cocktail, MD Biosciences) followed 24 hours later by an intra-peritoneal injection of LPS (K. Terato, et al., J. Immunology, 148: 2102-2108, 1992). In this model, the inflammation progresses rapidly and persists for 2 weeks culminating in ankylosis and permanent joint destruction.

In experiment A group of mice (n=5) were immunized with 50 μg Qβ VLP-IL-15 at day −70, day −56 and day −42, group of mice received PBS only was the negative control. In experiment B group of mice were immunized with 25 μg Qβ VLP-IL-15 day −42, day −28 and day −14 and group of mice immunized with Qβ only was the negative control. After three times' immunization, RA was induced in the mice at day 0 by injecting intravenously 2 mg of monoclonal antibody cocktail (Arthrogenic Monoclonal Antibody Cocktail, MD Biosciences) and 24 hours later with 200 μl of LPS. The inflammatory process was monitored over 14-15 days and the clinical scores were assigned to each limb. Clinical scores of arthritis were measured over 15 days. Clinical scores from 0 to 3 were assigned to each limb according to the following definitions: 0 normal, 1 mild erythema and/or swelling of digits/paw, 2 erythema and swelling extending over whole paw/joint, 3 strong swelling, deformation of paw/joint, with ankylosis. Averages of 5 mice per group are given with standard errors of mean.

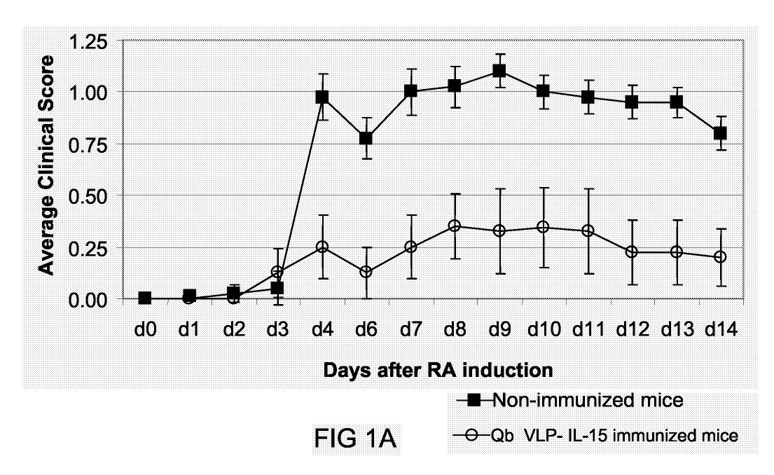

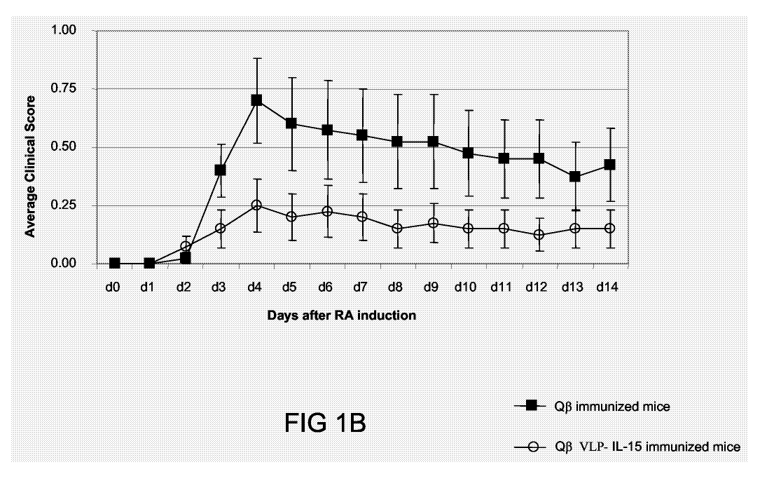

FIG. 1A shows the result of experiment A. Mice vaccinated with the Qβ VLP-IL-15 developed an average clinical score of approximately 0.25. In contrast, mice injected with PBS developed an average clinical score of 0.97 over the same period. FGI. 1B shows result of experiment B. Mice vaccinated with the Qβ VLP-IL-15 developed an average clinical score of 0.18, whereas the control mice had an average value of 0.51.

### Example 12

### Efficacy of Qβ VLP-IL-15 Vaccine in a Mouse Model of Atherosclerosis

Seven to eight weeks old male Apoe^{-/-} mice (The Jackson Laboratory, Bar Harbor Me.) were injected subcutaneously with either 50 μg Qβ-IL-15 vaccine (n=6) (obtained from EXAMPLE 7) or with 50 μg Qβ (n=6) on day 0, 14, 28, 49, 63 and 113. The mice were fed initially with a normal chow diet, which was replaced on day 21 by a western diet (20% fat, 0.15% cholesterol, Provimi Kliba AG). Mice were bled at regular intervals throughout the experiment and the antibody response against IL-15 was measured in the sera. Sacrifice was on day 159, and the aorta was isolated and prepared essentially as described (Tangirala R. K. et al. (1995) J. Lipd. Res. 36: 2320-2328). The animals were bled by cardiac puncture and perfused with cold PBS. The aorta was then exposed, as much of the adventitia removed in situ, and the aorta finally removed from the heart. The aorta was further cleaned from residual adventitia on a glass petri dish filled with cold PBS, and the arch of the aorta was sectioned 5 mm down from the left sub clavian artery. The aorta were cut longitudinally, pinned out on a black wax surface and fixed overnight in 4% formalin. They were then stained overnight in oil red O. The plaques were quantified with an imaging software (Motic Image Plus 2.0) on digital photographs. The plaque load was expressed as the sum of the surface of all plaques of the aorta taken up to the iliac bifurcation, divided by the total surface of the aorta measured up to the iliac bifurcation, in percentage. The difference in mean or median of the plaque load between the Qβ-IL-15 and Qβ group was analysed.

The antibody response was measured in a classical ELISA, with recombinant IL-15 coated on the ELISA plate. Binding of specific antibodies was detected using a goat anti-mouse HRP conjugate. The titers against IL-15 on day 0, 14, 28, 56 and 102 were calculated as the serum dilution giving half-maximal binding in the assay.

The extent of atherosclerosis in each animal is further evaluated by histological analysis of cross-sections through the aortic origin, as described by Ludewig B. et al. (2000) PNAS 97:12752-12757. Frozen serial cross-sections through the aortic origin are harvested beginning with the appearance of all three valve cusps. They are stained with oil red O and counter stained with hematoxylin to quantify lesion size.

The results of the measurement of the antibody response are shown in TABLE 2, and clearly demonstrate that immunisation against murine IL-15 coupled to Qβ led a strong specific antibody response against IL-15, since nearly no titer was detectable in the preimmune (d0) sera.

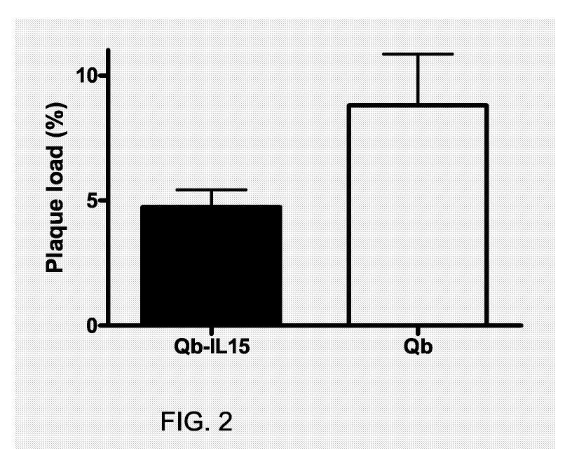

Furthermore, induction of an antibody response specific for IL-15 led to a reduction in mean (47%) and median (46%) plaque load in the QP-IL-15 group compared to the Qβ group (FIG. 2). This demonstrates that IL-15 is involved in the pathogenesis of atherosclerosis, and that induction of anti-IL-15 antibodies by the Qβ-IL-15 vaccine favorably modulates atherosclerosis.

TABLE 2
Geometric mean anti-IL-15 antibody titer in Apoe^{-/-} mice immunized with Qβ-IL-15

| | d0 | d14 | d21 | d28 | d42 | d49 | d63 | d92 | d159 |
|---|---|---|---|---|---|---|---|---|---|
| Mean | 10 ± 0 | 46 ± 27 | 2196 ± 13376 | 5767 ± 13007 | 25900 ± 19056 | 14355 ± 9978 | 48000 ± 31896 | 36707 ± 35521 | 84310 ± 39546 |

### Example 13

### Coupling of Mouse IL-15 Fragments to Qβ VLPs

Qβ virus like particle (2 mg/l) was derivatised with 2.8 mM SMPH (Pierce, Perbio Science) for 60 minutes at 25° C. and then dialysed against PBS. IL-15₆₁₋₇₃ (250 μM) and derivatised Qβ VLPs (100 μM) were incubated for one hours at 15° C. in PBS buffer. The coupling products were analysed by SDS-page. We identified the coupling product of one IL-15₆₁₋₇₃ molecule to one Qβ monomer and two IL-15₆₁₋₇₃ molecules to one Qβ monomer. IL-15₄₂₋₅₅ was also coupled to Qβ in a similar manner.

### Example 14

### Vaccine Efficacy in an Animal Model of Experimental Asthma

The effect of vaccination with Qβ-IL-15 in vivo is assessed in an ovalbumin (OVA) based murine model of asthma. This experiment tested the ability of the anti-IL-15 antibodies generated by vaccination with Qβ-IL-15 to down-regulate the in vivo action of endogenous IL-15. Six per group of BALB/c mice were analyzed in three groups. Mice were either vaccinated with 50 μg of Qβ-IL-15 (group C, obtained from EXAMPLE 7) or with Qβ VLP only (group A and B) as control on day 7, 21 and 35. High IgG titers against either Qβ or IL-15 were obtained after the second vaccination. Mice from group B and C were sensitised with 50 μg of OVA (grade V; Sigma-Aldrich) adsorbed to 2 mg of Al₂O₃ intraperitoneally on day 0. To induce a pulmonary allergic inflammation, these mice are challenged inhalationally with OVA aerosol (2.5% solution in PBS, 30 min nebulized with Pari TurboBOY; Pari) daily from day 42 to 45. As negative control, mice from group A were not treated with OVA and Al₂O₃ at day 0 and were not challenged with OVA aerosol subsequently. On day 46, mice are killed, a bronchoalveolar lavage (BAL) is performed, infiltrating cells in BAL are counted and airway hyperresponsiveness (AHR) is measured.

1. A composition comprising:
(a) a virus-like particle (VLP) with at least one first attachment site; and
(b) at least one antigen with at least one second attachment site, wherein said at least one antigen is an IL-15 protein, an IL-15 mutein or an IL-15 fragment and wherein (a) and (b) are linked through said at least one first and said at least one second attachment site.

2. The composition of claim 1, wherein said IL-15 protein comprises an amino acid sequence selected from the group consisting of:
(a) SEQ ID NO:22;
(b) SEQ ID NO:23;
(c) SEQ ID NO:24;
(d) SEQ ID NO:25; and
(e) an amino acid sequence which is at least 80%, preferably at least 85%, more preferably at least 90%, or most preferably at least 95% identical with any of SEQ ID NOs: 22-25.

3. The composition of claim 1, wherein said IL-15 mutein comprises an amino acid sequence selected from the group consisting of:
(a) SEQ ID NO:23, wherein position 46 is not E;
(b) SEQ ID NO:23, wherein position 50 is not I;
(c) SEQ ID NO:23, wherein position 46 is not E and position 50 is not I;
(d) SEQ ID NO:31;
(e) SEQ ID NO:32;
(f) SEQ ID NO:33; and
(g) an amino acid sequence which is at least 80%, preferably at least 85%, more preferably at least 90%, or most preferably at least 95% identical with SEQ ID NO:23 and wherein the position corresponding to position 46 of SEQ ID NO:23 is not E, or the position corresponding to position 50 of SEQ ID NO:23 is not 1, or the position corresponding to position 46 of SEQ ID NO:23 is not E and the position corresponding to position 50 of SEQ ID NO:23 is not I.

4. The composition of claim 1, wherein said IL-15 fragment comprises an amino acid sequence selected from the group consisting of:
(a) SEQ ID NO:34,
(b) SEQ ID NO:35;
(c) SEQ ID NO:36;
(d) SEQ ID NO:37;
(e) SEQ ID NO:38;
(f) SEQ ID NO:39; and
(g) An amino acid sequence which is at least 65%, preferably at least 80%, more preferably at least 85%, even more preferably at least 90%, or most preferably at least 95% identical with any of SEQ ID NO:34-39.

5. The composition of claim 1 wherein said VLP comprises recombinant coat proteins, mutants or fragments thereof, of a RNA-phage.

6. The composition of claim 5, wherein said RNA-phage is RNA-phage Qβ, fr, GA or AP205.

7. The composition of claim 1 wherein said first attachment site is linked to said second attachment site via at least one covalent bond, wherein preferably said covalent bond is a non-peptide bond.

8. The composition of claim 1 wherein said first attachment site comprises an amino group, preferably an amino group of a lysine.

9. The composition of claim 1 wherein said second attachment site comprises a sulfhydryl group, preferably a sulfhydryl group of a cysteine.

10. The composition of claim 1 claims further comprising a linker.

11. A vaccine comprises the composition of claim 1

12. The vaccine of claim 11, wherein said vaccine further comprises at least one adjuvant.

13. A method of immunization comprising administering said vaccine of claims 11 to an animal or a human.

14. A pharmaceutical composition comprising:
(a) the composition of claim 1 and
(b) an acceptable pharmaceutical carrier.

15. A method of producing the composition of claim 1 comprising:
(a) providing a VLP with at least one first attachment site;
(b) providing at least one antigen, wherein said antigen is an IL-15 protein, an IL-15 mutein or an IL-15 fragment, with at least one second attachment site; and
(c) linking said VLP and said at least one antigen to produce said composition, wherein said at least one antigen and said VLP are linked through said at least one first and said at least one second attachment sites.

16-24. (canceled)

25. A method of treating an inflammatory and/or chronic autoimmune disease comprising administering the composition of claim 1 to an animal or preferably to a human, wherein preferably said inflammatory and/or chronic autoimmune disease is rheumatoid arthritis.

26. A method of treating atherosclerosis comprising administering the composition of claim 1 to an animal or preferably to a human.

27. A method of treating asthma comprising administering the composition of claim 1 to an animal or preferably to a human.

28. A method of treating atherosclerosis or asthma comprising administering at least one IL-15 antagonist to an animal or preferably to a human.

29. The method of claim 28, wherein said IL-15 antagonist is a monoclonal antibody specifically binding to IL-15.

30. The method of claim 29, wherein said monoclonal antibody is produced in response to immunization with a composition comprising:
(a) a virus-like particle (VLP) with at least one first attachment site; and
(b) at least one antigen with at least one second attachment site, wherein said at least one antigen is an IL-15 protein, an IL-15 mutein or an IL-15 fragment and wherein (a) and (b) are linked through said at least one first and said at least one second attachment site.

31. The method of claim 28, wherein said IL-15 antagonist is an IL-15 mutein, wherein preferably said IL-15 mutein comprises an amino acid sequence as set forth in SEQ ID NO:23, wherein at least one position, preferably two, more preferably all three positions of Asp8, Gin101, and Gln108 of SEQ ID NO:23 is/are substituted.


SEQUENCE LISTING (55 sequences)

SEQ ID NO: 1 - protein, 132 aa, Bacteriophage Q-beta
AKLETVTLGNIGKDGKQTLVLNPRGVNPTNGVASLSQAGAVPALEKRVTVSVSQPSRNRKNYKVQVKIQNPTACTANGSCDPSVTRQAYADVTFSFTQYSTDEERAFVRTELAALLASPLLIDAIDQLNPAY

SEQ ID NO: 2 - protein, 329 aa, Bacteriophage Q-beta
MAKLETVTLGNIGKDGKQTLVLNPRGVNPTNGVASLSQAGAVPALEKRVTVSVSQPSRNRKNYKVQVKIQNPTACTANGSCDPSVTRQAYADVTFSFTQYSTDEERAFVRTELAALLASPLLIDAIDQLNPAYWTLLIAGGGSGSKPDPVIPDPPIDPPPGTGKYTCPFAIWSLEEVYEPPTKNRPWPIYNAVELQPREFDVALKDLLGNTKWRDWDSRLSYTTFRGCRGNGYIDLDATYLATDQAMRDQKYDIREGKKPGAFGNIERFIYLKSINAYCSLSDIAAYHADGVIVGFWRDPSSGGAIPFDFTKFDKTKCPIQAVIVVPRA

SEQ ID NO: 3 - protein, 129 aa, Bacteriophage R17
ASNFTQFVLVNDGGTGNVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGIY

SEQ ID NO: 4 - protein, 130 aa, Bacteriophage fr
MASNFEEFVLVDNGGTGDVKVAPSNFANGVAEWISSNSRSQAYKVTCSVRQSSANNRKYTVKVEVPKVATQVQGGVELPVAAWRSYMNMELTIPVFATNDDCALIVKALQGTFKTGNPIATAIAANSGIY

SEQ ID NO: 5 - protein, 130 aa, Bacteriophage GA
MATLRSFVLVDNGGTGNVTVVPVSNANGVAEWLSNNSRSQAYRVTASYRASGADKRKYAIKLEVPKIVTQVVNGVELPGSAWKAYASIDLTIPIFAATDDVTVISKSLAGLFKVGNPIAEAISSQSGFYA

SEQ ID NO: 6 - protein, 132 aa, Bacteriophage SP
MAKLNQVTLSKIGKNGDQTLTLTPRGVNPTNGVASLSEAGAVPALEKRVTVSVAQPSRNRKNFKVQIKLQNPTACTRDACDPSVTRSAFADVTLSFTSYSTDEERALIRTELAALLADPLIVDAIDNLNPAY

SEQ ID NO: 7 - protein, 329 aa, Bacteriophage SP
AKLNQVTLSKIGKNGDQTLTLTPRGVNPTNGVASLSEAGAVPALEKRVTVSVAQPSRNRKNFKVQIKLQNPTACTRDACDPSVTRSAFADVTLSFTSYSTDEERALIRTELAALLADPLIVDAIDNLNPAYWAALLVASSGGGDNPSDPDVPVVPDVKPPDGTGRYKCPFACYRLGSIYEVGKEGSPDIYERGDEVSVTFDYALEDFLGNTNWRNWDQRLSDYDIANRRRCRGNGYIDLDATAMQSDDFVLSGRYGVRKVKFPGAFGSIKYLLNIQGDAWLDLSEVTAYRSYGMVIGFWTDSKSPQLPTDFTQFNSANCPVQTVIIIPS

SEQ ID NO: 8 - protein, 130 aa, Bacteriophage MS2
MASNFTQFVLVDNGGTGDVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGIY

SEQ ID NO: 9 - protein, 133 aa, Bacteriophage M11
MAKLQAITLSGIGKKGDVTLDLNPRGVNPTNGVAALSEAGAVPALEKRVTISVSQPSRNRKNYKVQVKIQNPTSCTASGTCDPSVTRSAYSDVTFSFTQYSTVEERALVRTELQALLADPMLVNAIDNLNPAY

SEQ ID NO: 10 - protein, 133 aa, Bacteriophage MX1
MAKLQAITLSGIGKNGDVTLNLNPRGVNPTNGVAALSEAGAVPALEKRVTISVSQPSRNRKNYKVQVKIQNPTSCTASGTCDPSVTRSAYADVTFSFTQYSTDEERALVRTELKALLADPMLIDAIDNLNPAY

SEQ ID NO: 11 - protein, 330 aa, Bacteriophage NL95
MAKLNKVTLTGIGKAGNQTLTLTPRGVNPTNGVASLSEAGAVPALEKRVTVSVAQPSRNRKNYKVQIKLQNPTACTKDACDPSVTRSGSRDVTLSFTSYSTERERALIRTELAALLKDDLIVDAIDNLNPAYWAALLAASPGGGNNPYPGVPDSPNVKPPGGTGTYRCPFACYRRGELITEAKDGACALYACGSEALVEFEYALEDFLGNEFWRNWDGRLSKYDIETHRRCRGNGYVDLDASVMQSDEYVLSGAYDVVKMQPPGTFDSPRYYLHLMDGIYVDLAEVTAYRSYGMVIGFWTDSKSPQLPTDFTRFNRHNCPVQTVIVIPSL

SEQ ID NO: 12 - protein, 129 aa, Bacteriophage f2
ASNFTQFVLVNDGGTGNVTVAPSNFANGVAEWISSNSRSQAYKVTCSVRQSSAQNRKYTIKVEVPKVATQTVGGVELPVAAWRSYLNLELTIPIFATNSDCELIVKAMQGLLKDGNPIPSAIAANSGIY

SEQ ID NO: 13 - protein, 128 aa, Bacteriophage PP7
MSKTIVLSVGEATRTLTEIQSTADRQIFEEKVGPLVGRLRLTASLRQNGAKTAYRVNLKLDQADVVDCSTSVCGELPKVRYTQVWSHDVTIVANSTEASRKSLYDLTKSLVATSQVEDLVVNLVPLGR

SEQ ID NO: 14 - protein, 131 aa, bacteriophage AP205
MANKPMQPITSTANKIVWSDPTRLSTTFSASLLRQRVKVGIAELNNVSGQYVSVYKRPAPKPEGCADACVIMPNENQSIRTVISGSAENLATLKAEWETHKRNVDTLFASGNAGLGFLDPTAAIVSSDTTA

SEQ ID NO: 15 - protein, 132 aa, Artificial Sequence
  Bacteriophage Qbeta 240 mutant
AKLETVTLGNIGRDGKQTLVLNPRGVNPTNGVASLSQAGAVPALEKRVTVSVSQPSRNRKNYKVQVKIQNPTACTANGSCDPSVTRQKYADVTFSFTQYSTDEERAFVRTELAALLASPLLIDAIDQLNPAY

SEQ ID NO: 16 - protein, 132 aa, Artificial Sequence
  Bacteriophage Q-beta 243 mutant
AKLETVTLGKIGKDGKQTLVLNPRGVNPTNGVASLSQAGAVPALEKRVTVSVSQPSRNRKNYKVQVKIQNPTACTANGSCDPSVTRQKYADVTFSFTQYSTDEERAFVRTELAALLASPLLIDAIDQLNPAY

SEQ ID NO: 17 - protein, 132 aa, Artificial Sequence
  Bacteriophage Q-beta 250 mutant
ARLETVTLGNIGRDGKQTLVLNPRGVNPTNGVASLSQAGAVPALEKRVTVSVSQPSRNRKNYKVQVKIQNPTACTANGSCDPSVTRQKYADVTFSFTQYSTDEERAFVRTELAALLASPLLIDAIDQLNPAY

SEQ ID NO: 18 - protein, 132 aa, Artificial Sequence
  Bacteriophage Q-beta 251 mutant
AKLETVTLGNIGKDGRQTLVLNPRGVNPTNGVASLSQAGAVPALEKRVTVSVSQPSRNRKNYKVQVKIQNPTACTANGSCDPSVTRQKYADVTFSFTQYSTDEERAFVRTELAALLASPLLIDAIDQLNPAY

SEQ ID NO: 19 - protein, 132 aa, Artificial Sequence
  Bacteriophage Q-beta 259 mutant
ARLETVTLGNIGKDGRQTLVLNPRGVNPTNGVASLSQAGAVPALEKRVTVSVSQPSRNRKNYKVQVKIQNPTACTANGSCDPSVTRQKYADVTFSFTQYSTDEERAFVRTELAALLASPLLIDAIDQLNPAY

SEQ ID NO: 20 - protein, 185 aa, Hepatitis B virus
MDIDPYKEFGATVELLSFLPSDFFPSVRDLLDTASALYREALESPEHCSPHHTALRQAILCWGELMTLATWVGNNLEDPASRDLVVNYVNTNMGLKIRQLLWFHISCLTFGRETVLEYLVSFGVWIRTPPAYRPPNAPILSTLPETTVVRRRDRGRSPRRRTPSPRRRRSQSPRRRRSQSRESQC

SEQ ID NO: 21 - protein, 188 aa, Hepatitis B virus
MDIDPYKEFGATVELLSFLPSDFFPSVRDLLDTAAALYRDALESPEHCSPHHTALRQAILCWGDLMTLATWVGTNLEDGGKGGSRDLVVSYVNTNVGLKFRQLLWFHISCLTFGRETVLEYLVSFGVWIRTPPAYRPPNAPILSTLPETTVVRRRDRGRSPRRRTPSPRRRRSQSPRRRRSQSRESQC

SEQ ID NO: 22 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 23 - protein, 114 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 24 - protein, 114 aa, Mus musculus
NWIDVRYDLEKIESLIQSIHIDTTLYTDSDFHPSCKVTAMNCFLLELQVILHEYSNMTLNETVRNVLYLANSTLSSNKNVAESGCKECEELEEKTFTEFLQSFIRIVQMFINTS

SEQ ID NO: 25 - protein, 114 aa, Rattus norvegicus
NWIDVRYDLEKIESLIQFIHIDTTLYTDSDFHPSCKVTAMNCFLLELQVILHEYSNMTLNETVRNVLYLANSTLSSNKNVIESGCKECEELEERNFTEFLQSFIHIVQMFINTS

SEQ ID NO: 26 - DNA, 64 bp, artificial sequence
  primer to change restriction sites
gatccgctagccctcgaggactacaaggatgacgacgacaagggtggttgcggttaataagttt

SEQ ID NO: 27 - DNA, 60 bp, artificial sequence
  primer to change restriciton sites
aaacttattaaccgcaaccacccttgtcgtcgtcatccttgtagtcctcgagggctagcg

SEQ ID NO: 28 - DNA, 33 bp, artificial sequence
  primer to clone IL-15
ggaattccatatgaactggatagatgtaagata

SEQ ID NO: 29 - DNA, 31 bp, artificial sequence
  primer to clone IL-15
cccgctcgagggacgtgttgatgaacatttg

SEQ ID NO: 30 - protein, 128 aa, Mus musculus
NWIDVRYDLEKIESLIQSIHIDTTLYTDSDFHPSCKVTAMNCFLLELQVILHEYSNMTLNETVRNVLYLANSTLSSNKNVAESGCKECEELEEKTFTEFLQSFIRIVQMFINTSLEDYKDDDDKGGCG

SEQ ID NO: 31 - protein, 114 aa, artificial sequence
  human IL-15 E46K
NWVNVISDLKKIEDLIQSMHIDATLYTESDVHPSCKVTAMKCFLLKLQVISLESGDASIHDTVENLIILANNSLSSNGNVTESGCKECEELEEKNIKEFLQSFVHIVQMFINTS

SEQ ID NO: 32 - protein, 114 aa, artificial sequence
  human IL-15 I50D
NWVNVISDLKKIEDLIQSMHIDATLYTESDVHPSCKVTAMKCFLLELQVDSLESGDASIHDTVENLIILANNSLSSNGNVTESGCKECEELEEKNIKEFLQSFVHIVQMFINTS

SEQ ID NO: 33 - protein, 114 aa, artificial sequence
  Human IL-15 E46K, I50D
NWVNVISDLKKIEDLIQSMHIDATLYTESDVHPSCKVTAMKCFLLKLQVDSLESGDASIHDTVENLIILANNSLSSNGNVTESGCKECEELEEKNIKEFLQSFVHIVQMFINTS

SEQ ID NO: 34 - protein, 13 aa, artificial sequence
  a fragment of IL-15
FLLELQVILHEYS

SEQ ID NO: 35 - protein, 13 aa, Mouse cytomegalovirus 1
ETVRNVLYLANST

SEQ ID NO: 36 - protein, 13 aa, artificial sequence
  human fragment 42-55
FLLELQVISLESG

SEQ ID NO: 37 - protein, 13 aa, artificial sequence
  human fragment 61-73
DTVENLIILANNS

SEQ ID NO: 38 - protein, 13 aa, artificial sequence
  human 42-55 E46K
FLLKLQVISLESG

SEQ ID NO: 39 - protein, 13 aa, artificial sequence
  human fragment 42-55 I50D
FLLELQVDSLESG

SEQ ID NO: 40 - protein, 13 aa, artificial sequence
  human fragment 42-55 E46K I50D
FLLKLQVDSLESG

SEQ ID NO: 41 - protein, 176 aa, artificial sequence
  IL-15 soluble receptor
GITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTT

SEQ ID NO: 42 - protein, 114 aa, artificial sequence
  IL-15 mutein Q101D, Q108D
NWVNVISDLKKIEDLIQSMHIDATLYTESDVHPSCKVTAMKCFLLELQVISLESGDASIHDTVENLIILANNSLSSNGNVTESGCKECEELEEKNIKEFLDSFVHIVDMFINTS

SEQ ID NO: 43 - DNA, 82 bp, artificial sequence
  cloning site of the vector
catatggatccgctagccctcgaggactacaaggatgacgacgacaagggtggttgcggttaataagtttaaacgcggccgc

SEQ ID NO: 44 - protein, 10 aa, artificial sequence
  gamma linker 1
CGDKTHTSPP

SEQ ID NO: 45 - protein, 6 aa, artificial sequence
  N terminial glycine linker
GCGGGG

SEQ ID NO: 46 - protein, 6 aa, artificial sequence
  N-terminal glycine serine linker
CGGGGS

SEQ ID NO: 47 - protein, 9 aa, artificial sequence
  GCGSGGGGS linker
GCGSGGGGS

SEQ ID NO: 48 - protein, 10 aa, artificial sequence
  C-terminal gamma 1 linker
DKTHTSPPCG

SEQ ID NO: 49 - protein, 18 aa, artificial sequence
  C terminal gamma linker 3
PKPSTPPGSSGGAPGGCG

SEQ ID NO: 50 - protein, 6 aa, artificial sequence
  C terminal glycine linker
GGGGCG

SEQ ID NO: 51 - protein, 6 aa, artificial sequence
  C terminal glycine serine linker
SGGGGC

SEQ ID NO: 52 - protein, 10 aa, artificial sequence
  GSGGGGSGCG linker
GSGGGGSGCG

SEQ ID NO: 53 - protein, 6 aa, artificial sequence
  glycine lysine linker
GGKKGC

SEQ ID NO: 54 - protein, 6 aa, artificial sequence
  glycine lysine linker 2
CGKKGG

SEQ ID NO: 55 - protein, 17 aa, artificial sequence
  N-terminal gamma 3-linker
CGGPKPSTPPGSSGGAP